(19) United States
(12) Patent Application Publication
(10) Pub. No.: US 2026/0130891 A1
(43) Pub. Date: May 14, 2026

(54) PIM KINASE INHIBITOR IN COMBINATION WITH KRAS INHIBITOR

(71) Applicants: NINGBO NEWBAY TECHNOLOGY DEVELOPMENT CO., LTD, Ningbo, Zhejiang (CN); SHANGHAI NEWBAY MEDICAL TECHNOLOGY CO., LTD, Shanghai (CN)

(72) Inventors: Yanhua Xu, Shanghai (CN); Kui Lin, Shanghai (CN); Zhenhai Shen, Ningbo, Zhejiang (CN)

(21) Appl. No.: 19/121,771

(22) PCT Filed: Jul. 13, 2023
(86) PCT No.: PCT/CN2023/107140

§ 371 Date: Apr. 16, 2025

(30) Foreign Application Priority Data
Oct. 17, 2022 (WO) PCT/CN2022/125786

(51) Int. Cl. A61K 31/427; A61K 31/416; A61K 31/4439; A61K 31/444; A61K 31/454; A61K 31/4545; A61K 31/496; A61K 31/517; A61K 31/519; A61K 31/5386; A61K 31/55; A61K 31/553; A61P 35/00

(52) U.S. Cl. CPC A61K 31/427; A61K 31/416; A61K 31/4439; A61K 31/444; A61K 31/454; A61K 31/4545; A61K 31/496; A61K 31/517; A61K 31/519; A61K 31/5386; A61K 31/55; A61K 31/553; A61P 35/00

(57) ABSTRACT

It relates to the combination of PIM kinase inhibitor and a KRAS inhibitor for use in treating cancer, in a subject in need thereof. Also provided are compositions or kits comprising same. Furthermore, it also relates to a PIM kinase inhibitor for use in treating cancer with KRAS mutation in a human subject in need thereof.

### FIELD OF THE INVENTION

The present invention provides combination therapy that includes a PIM kinase inhibitor and a KRAS inhibitor for the treatment of cancer. The invention also relates to pharmaceutical compositions or kits comprising a PIM kinase inhibitor and a KRAS inhibitor for the treatment of cancers. The invention also relates to a PIM kinase inhibitor for use in treating cancer with KRAS mutation.

### BACKGROUND

KRAS gene mutations are common in pancreatic cancer, lung adenocarcinoma, colorectal cancer (CRC), gallbladder cancer, thyroid cancer, and bile duct cancer. Among KRAS mutations, the G12 codon (81%) is the most frequently mutated, followed by G13 (14%) and Q61 (2%). KRAS mutations are the most common RAS mutations in pancreatic cancer (88%), followed by colon adenocarcinoma (50%), rectal adenocarcinoma (50%), lung adenocarcinoma (32%), small intestine adenocarcinoma (26%), cholangiocarcinoma (23%), plasma cell myeloma (18%), gallbladder carcinoma (16%), and anaplastic thyroid carcinoma (8.6%) (Kwan et al. J Exp Clin Cancer Res (2022) 41:27). Accordingly, there is a strong interest in agents that block the proliferative signaling induced by the oncogenic KRAS variants.

While KRAS was regarded as an undruggable target for decades, in 2013, investigators identified a hidden pocket next to the mutant cysteine in the KRAS G12C protein that was only revealed in the GDP-bound form, which finally offered a direct drug-binding site. Various attempts have been made to develop KRAS inhibitors. In May 2021, AMG510 (sotorasib) became the first FDA-approved therapy to directly target KRAS-mutated tumors. In June 2021, MRTX849 (adagrasib) received breakthrough therapy designation by the FDA. Additional KRAS inhibitors are currently ongoing, such as ARS-853, ARS-1620, ARS-3248(JNJ-74699157), MRTX1257, LY3499446, LY3537982, BI 1823911, GDC-6036, RMC-6291, RMC-6236, AZD4625, D-1553, JDQ443, MRTX1133, BI 1701963 and BAY-293.

Despite the clinical benefit that was observed for many patients treated with KRAS^{G12C} inhibitors adagrasib or sotorasib, acquired resistance to single-agent therapy eventually occurred in most patients. Patients with KRAS mutant tumors have significantly poorer outcomes and worse prognosis. Effective combination therapy appears to be necessary to overcome this acquired resistance to direct KRAS^{G12C} inhibitors. WO2020106647A2 discloses KRAS^{G12C} inhibitors in combination with carboplatin, anti-PD-1 inhibitor, MEK inhibitor, EGFR inhibitor, TOR inhibitor, SHP2 inhibitor, P13K inhibitor or AKT inhibitor. JANE DE LARTIGUE also discloses KRAS^{G12C} inhibitors in combination with pan-ERBB inhibitor, CDK4/6 inhibitor, SOS1/pan-KRAS inhibitor (Jane de Lartigue. OncologyLive, Vol. 23/No. 1, Volume 23, Issue 01. table).

Moreover, several MAPK-pathway targeted therapies are contra-indicated for treatment of KRAS mutant tumors due to lack of clinical efficacy. Additionally, non-tumor or non-mutant selective therapies can introduce on-target toxicities due to inhibition of MAPK signaling in normal cells. This might limit the utility for combining such agents with standard-of-care or immunotherapy. Thus, there exists a significant unmet need for the development of tumor-selective therapies that do not introduce liabilities for normal cells. Until now, there is no study of the combination therapy of PIM inhibitor and KRAS inhibitors, especially the combination of PIM inhibitors and KRAS^{G12C} inhibitors, KRAS^{G12D} inhibitors, or KRAS^{G12V} inhibitors.

### SUMMARY OF THE INVENTION

In order to overcome the resistance to KRAS inhibitors over extended treatment times, the present disclosure provides a combination therapy that includes a PIM kinase inhibitor (PIMi) and a KRAS inhibitor. It was found that resistance to KRAS inhibitors could be reversed by co-treatment with PIM kinase inhibitors, especially the acquired resistance. Furthermore, it was found that the combination of a PIM kinase inhibitor with a KRAS inhibitor showed superior efficacy relative to either of the monotherapy treatments, and exhibited synergy against KRAS mutant cancer. Additionally, it was found that PIM inhibitor GDC-0570 could be used as a single active agent in various KRAS mutant cancers.

Based on these discoveries, the present disclosure is related to the following aspects.

In one aspect, the present disclosure provides a method for treating cancer in a subject in need thereof, comprising administering therapeutically effective amounts of a PIM kinase inhibitor and a KRAS inhibitor to the subject. In certain embodiments, the cancer is a KRAS mutant cancer or KRAS inhibitor resistant cancer.

In another aspect, the present disclosure provides the use of the combination of a PIM kinase inhibitor and a KRAS inhibitor in the treatment of cancer, such as KRAS mutant cancer or KRAS inhibitor resistant cancer.

In another aspect, the present disclosure provides the use of the combination of a PIM kinase inhibitor and a KRAS inhibitor in the manufacture of a medicament for use in the treatment of cancer, such as KRAS mutant cancer or KRAS inhibitor resistant cancer.

In another aspect, the present disclosure provides a pharmaceutical composition comprising a PIM kinase inhibitor and a KRAS inhibitor. Preferably, the pharmaceutical composition is for use in the treatment of cancer, such as KRAS mutant cancer or KRAS inhibitor resistant cancer.

In another aspect, the present disclosure provides a kit comprising a PIM kinase inhibitor, a KRAS inhibitor and a pharmaceutical acceptable excipient.

In yet another aspect, the present disclosure provides a method for treating cancer with KRAS mutation in a human subject in need thereof, comprising administering therapeutically effective amounts of N-(5-((2S,5R,6S)-5-amino-6-fluorooxepan-2-yl)-1-methyl-1H-pyrazol-4-yl)-2-(2,6-difluorophenyl)thiazole-4-carboxamide (GDC-0570) or a pharmaceutically acceptable salt thereof. The GDC-0570 could be used as a single active agent in various KRAS mutant cancers.

### BRIEF DESCRIPTION OF THE DRAWINGS

FIG. 1 shows the effect of GDC-0570 and sotorasib on tumor volume in KRAS G12C mutant CRC PDX model CRC024.

FIG. 2 shows the effect of GDC-0570 and sotorasib on tumor volume in KRAS G12C mutant NSCLC PDX model LUN055.

FIG. 3 shows the effect of GDC-0570 and sotorasib on tumor volume in KRAS G12C mutant NSCLC PDX model LUN156.

FIG. 4 shows the effect of GDC-0570 and sotorasib on tumor volume in KRAS G12C mutant NSCLC PDX model LUN2156-44.

FIG. 5 shows the effect of GDC-0570 and GDC-6036 on tumor volume in KRAS G12C mutant CRC PDX model CRC022.

FIG. 6 shows the effect of GDC-0570 on tumor volume in KRAS G12C mutant NSCLC PDX model LUN156.

FIG. 7 shows the effect of GDC-0570 on tumor volume in KRAS G12D mutant NSCLC PDX model LUN #137.

FIG. 8 shows the effect of GDC-0570 and cobimetinib on tumor volume in KRAS G12D mutant pancreatic PDX model PAN092.

FIG. 9 shows the effect of GDC-0570 and MRTX1133 on tumor volume in KRAS G12D pancreatic cancer models PAN092.

FIG. 10 shows the effect of GDC-0570 and cobimetinib on tumor volume in KRAS G12V mutant CRC PDX models.

### DEFINITIONS

Unless defined otherwise, technical and scientific terms used herein have the same meaning as commonly understood by one of ordinary skill in the art to which this disclosure pertains.

The words “comprise,” “comprising,” “include,” “including,” and “includes” are intended to specify the presence of stated features, integers, components, or steps, but they do not preclude the presence or addition of one or more other features, integers, components, steps, or groups thereof.

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The term “about” or “approximately” usually means within 5%, or more preferably within 1%, of a given value or range.

The recitation of numerical ranges by endpoints includes all numbers and fractions subsumed within the respective ranges, as well as the recited endpoints.

The terms “treat”, “treating”, and “treatment” refer to therapeutic treatment in a subject having cancer, wherein the object is to prevent or slow down (lessen) an undesired physiological change or disorder, such as the growth, development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total). “Treatment” can also mean prolonging survival as compared to expected survival if not receiving treatment.

The phrase “therapeutically effective amount” means a combined amount of the PIM kinase inhibitor and KRAS inhibitor that (i) treats the cancer, (ii) attenuates, ameliorates, or eliminates one or more symptoms of the cancer, and/or (iii) prevents or delays the onset of one or more symptoms of the cancer, wherein the combined amount has demonstrated an improvement in (i), (ii), or (iii) compared to single agent therapy. The therapeutically effective amount of the combination may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the combination may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “synergistic” as used herein refers to a therapeutic combination which is more effective than the additive effects of the two or more single agents. A determination of a synergistic interaction between the PIM kinase inhibitor and the KRAS inhibitor may be based on the results obtained from the assays described herein. The combination therapy may provide “synergy” and prove “synergistic”, i.e., the effect achieved when the active ingredients used together is greater than the sum of the effects that results from using the compounds separately. A synergistic effect may be attained, in one example, when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes or by different oral doses. In general, during alternation therapy, an effective dosage of each active ingredient is administered sequentially, i.e., serially, whereas in combination therapy, effective dosages of two or more active ingredients are administered together. Synergy may be evaluated by the tumor growth inhibition. Specifically, the tumor volume growth trend is suppressed, and preferably the tumor volume is superiorly shrunk, or the tumor is in complete regression.

“Resistant cancer” or “refractory cancer” is used interchangeably herein, and refers to a cancer that is not responsive or less responsive to therapeutic treatment. Resistant cancer may have intrinsic resistance, acquired resistance or adaptive resistance. “Intrinsic resistance” or “Primary resistance” means a lack of tumor response to initial therapy. “Acquired resistance” refers to tumors that initially respond to treatment and later relapsed. “Adaptive resistance” refers to resistance induced by alterations of the upstream or downstream or parallel pathway components of KRAS-mutant cancer, inevitably resulting in a lack of efficacy, and recurrence and progression of these tumors. For example, “KRAS resistant cancer” comprises cancers that have acquired resistance to one or more KRAS inhibitors by prior treatment with one or more KRAS inhibitors, or may comprise cancers with intrinsic resistance to one or more KRAS inhibitors, such as cancers having an activated PIM kinase.

The resistant cancer may be resistant at the beginning of treatment, or it may become resistant during treatment.

The term “sensitive cancer” as used herein refers to a cancer that responds to a certain drug treatment without progression. For example, the sensitive cancer is sotorasib-sensitive KRAS G12C mutant colorectal cancer or sotorasib-sensitive KRAS G12C mutant NSCLC cancer. Administrating 100 mg/kg sotorasib to those cancer PDX models results in >100% TGI.

The term “less sensitive cancer” as used herein refers to a cancer that progresses on a certain drug treatment. For example, the less sensitive cancer is sotorasib-less sensitive KRAS G12C mutant NSCLC cancer. Administrating 100 mg/kg sotorasib to this cancer PDX model results in <100% TGI.

The term “combination” refers to simultaneous, separate or sequential administration. Where the administration is sequential or separate, the delay in administering the second component should not be such as to lose the beneficial effect of the combination.

As used herein, the subject is a mammal. In some embodiments, the subject is a human. In some embodiments, the subject is an adult human subject. In some embodiments, the subject is a human male subject. In some embodiments, the subject is a human female subject. “Subject” and “patient” and “individual” are also used interchangeably herein.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” refers to pharmaceutically acceptable organic or inorganic salts of a compound. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. If the compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18th ed., (1995) Mack Publishing Co., Easton PA; and in The Orange Book (Food & Drug Administration, Washington, D.C. on their website). If the compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

All publications, published patent documents, and patent applications cited herein are hereby incorporated by reference to the same extent as though each individual publication, published patent document, or patent application was specifically and individually indicated as being incorporated by reference.

### DETAILED DESCRIPTION

### 1. Exemplary PIM Kinase Inhibitors and a KRAS Inhibitors

The present disclosure is generally related to the combination of a PIM kinase inhibitor and a KRAS inhibitor a as described herein, e.g., for use in the treatment of cancer.

In some embodiments, the PIM kinase inhibitor is a PIM-1 kinase inhibitor, PIM-2 kinase inhibitor, or PIM-3 inhibitor. In some embodiments, the PIM kinase inhibitor is a pan-PIM kinase inhibitor, which exhibits potent activity against PIM-1, PIM-2 and/or PIM-3 inhibitor. Exemplary PIM kinase inhibitors include, but are not limited to, AZD1208, LGH447, and the compounds disclosed in WO2014048939, US20110059961 or US20130079321 (such as GDC-0570, GNE-1571, GNE-5775, GDC-0339 and GNE-5652), and pharmaceutically acceptable salts thereof, the structures of which are provided below:

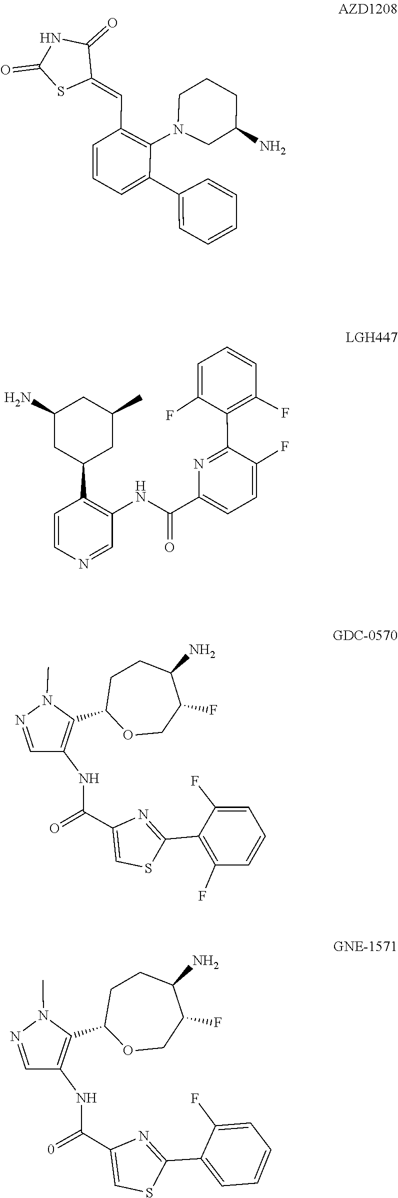

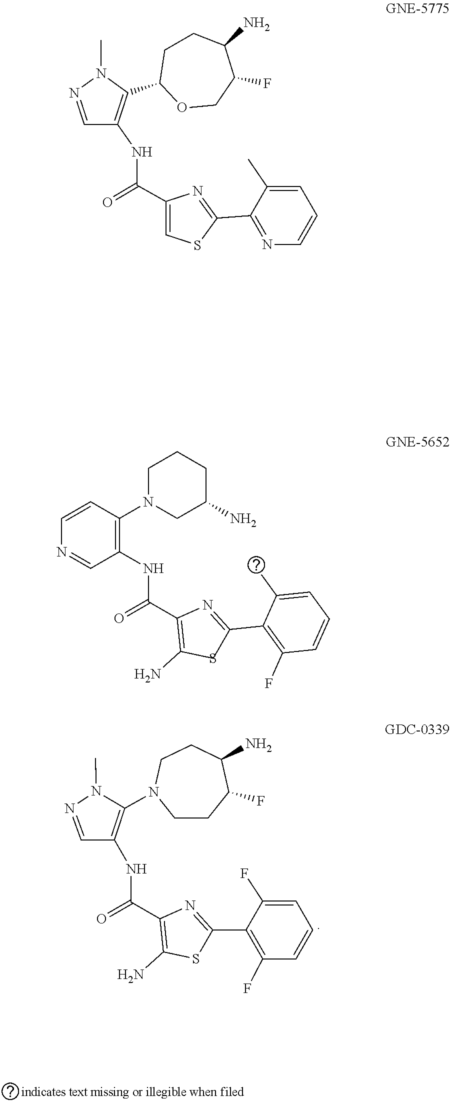 [SMILES: Cc1cccnc1-c1nc(C(=O)Nc2cnn(C)c2[C@@H]2CC[C@@H](N)[C@H](F)CO2)cs1 ; Cc1cccc(F)c1-c1nc(C(=O)Nc2cnccc2N2CCC[C@H](N)C2)c(N)s1 ; Cn1ncc(NC(=O)c2nc(-c3c(F)cccc3F)sc2N)c1N1CC[C@@H](N)[C@H](F)CC1]

In some embodiments, the PIM kinase inhibitor is selected from the group consisting of GDC-0570, GNE-1571, GNE-5775, GDC-0339, GNE-5652, and pharmaceutically acceptable salts thereof.

In some embodiments, the PIM kinase inhibitor is GDC-0570, also known as N-(5-((2S,5R,6S)-5-amino-6-fluorooxepan-2-yl)-1-methyl-1H-pyrazol-4-yl)-2-(2,6-difluoroph enyl)thiazole-4-carboxamide, or a pharmaceutically acceptable salt thereof. GDC-0570 is Compound 321 in WO2014048939.

In some embodiments, the PIM kinase inhibitor is GNE-1571, also known as N-(5-((2S,5R,6S)-5-amino-6-fluorooxepan-2-yl)-1-methyl-1H-pyrazol-4-yl)-2-(2-fluoropheny 1)thiazole-4-carboxamide, or a pharmaceutically acceptable salt thereof. GNE-1571 is Compound 322 in WO2014048939.

In some embodiments, the PIM kinase inhibitor is GNE-5775, also known as N-(5-((2S,5R,6S)-5-amino-6-fluorooxepan-2-yl)-1-methyl-1H-pyrazol-4-yl)-2-(3-methylpyridin-2-yl)thiazole-4-carboxamide, or a pharmaceutically acceptable salt thereof. GNE-5775 is Compound 231 in WO2014048939.

In some embodiments, the PIM kinase inhibitor is GDC-0339, also known as 5-amino-N-(5-((4R,5R)-4-amino-5-fluoroazepan-1-yl)-1-methyl-1H-pyrazol-4-yl)-2-(2,6-difl uorophenyl)thiazole-4-carboxamide, or a pharmaceutically acceptable salt thereof. GDC-0339 is the compound of Example 139 in US20130079321.

In some embodiments, the PIM kinase inhibitor is GNE-5652, also known as (S)-5-amino-N-(4-(3-aminopiperidin-1-yl)pyridin-3-yl)-2-(2,6-difluorophenyl) thiazole-4-carboxamide, or a pharmaceutically acceptable salt thereof. GNE-5652 is the compound of Example 3 in US20110059961.

In some embodiments, the KRAS inhibitor is KRAS^{G12C} inhibitor, KRAS^{G12V} inhibitor, KRAS^{G12D} inhibitor, KRAS^{G13C} inhibitor, KRAS^{G13D} inhibitor, KRAS^{Q61H} inhibitor, KRAS^{Q61L} inhibitor, KRAS^{Q61R} inhibitor, KRAS^{KH7N} inhibitor, pan-KRAS inhibitor, KRAS-SOS1 interaction inhibitor, or KRAS signaling inhibitor, and pharmaceutically acceptable salts thereof.

In some embodiments, the KRAS^{G12C} inhibitor is sotorasib (AMG 510), ARS-853, ARS-1620, ARS-3248(JNJ-74699157), adagrasib (MRTX849), MRTX1257, LY3499446, LY3537982, BI 1823911, RG6330 (GDC-6036), RMC-6291, RMC-6236, AZD4625, D-1553, JDQ443, MK-1084, and pharmaceutically acceptable salts thereof. The structures of some KRAS^{G12C} inhibitor are provided below:

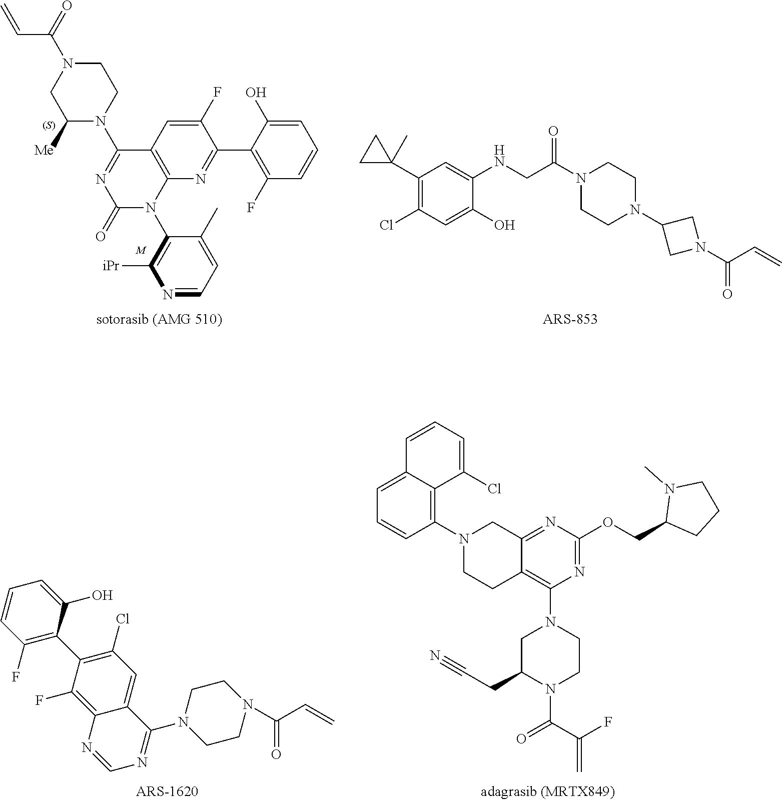

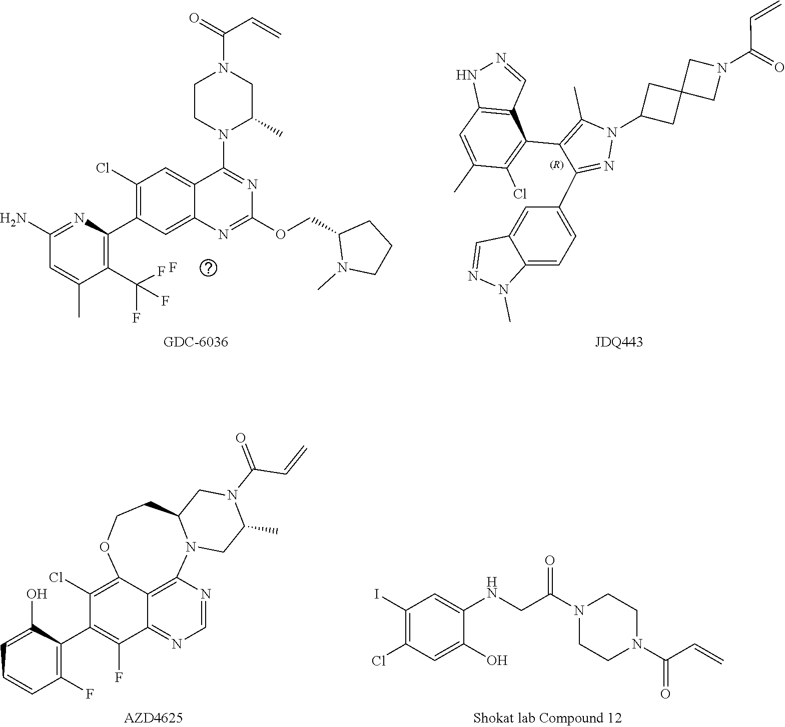

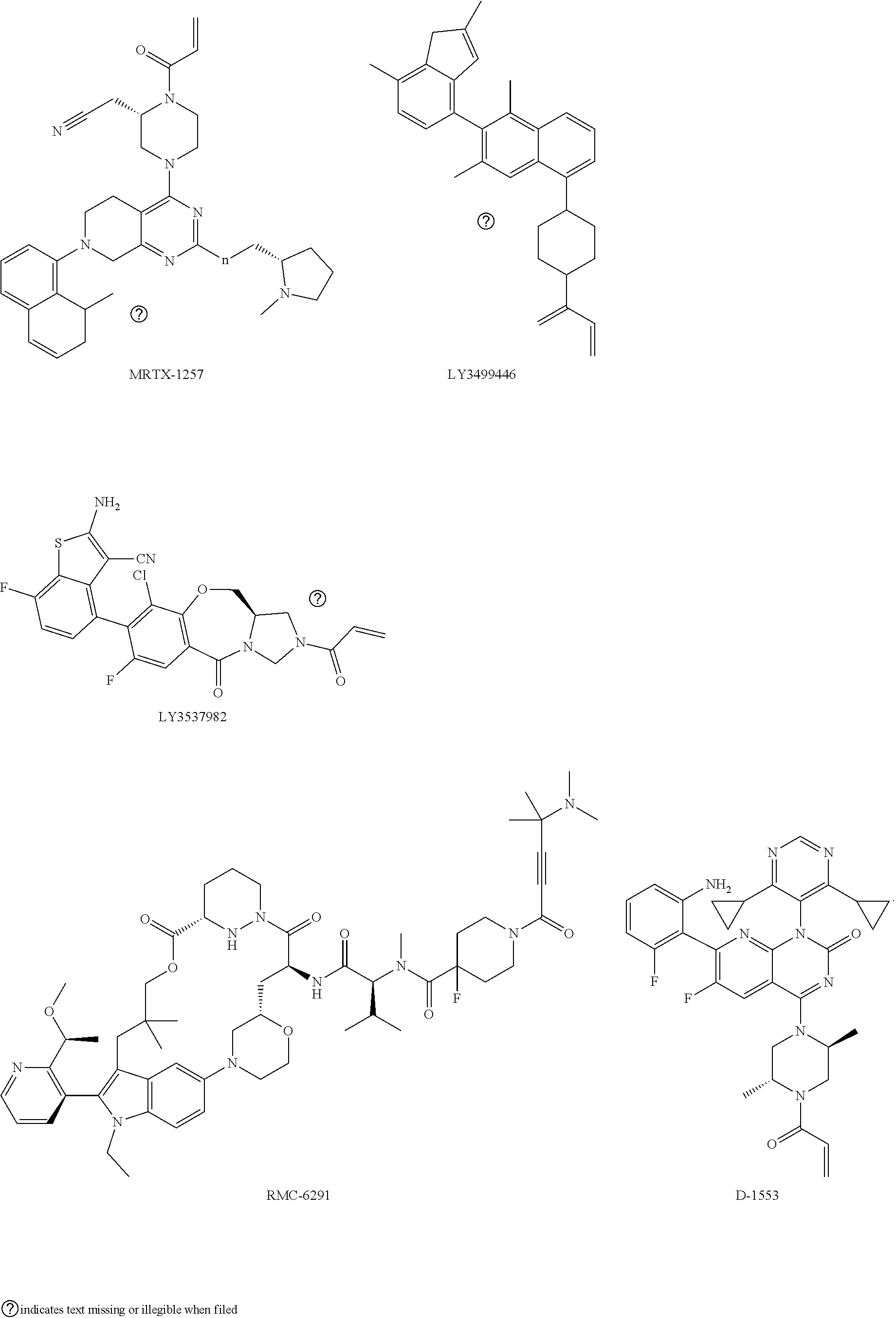

In some embodiments, the KRAS^{G12C} inhibitors are disclosed in WO2014152588, WO2015054572, WO2016049524, WO2016164675, WO2016168540, WO2017015562, WO2017058915, WO2017058807, WO2017058792, WO2017058902, WO2017087528, WO2017201161, WO2018064510, WO2018068017, WO2018119183, WO2018140600, WO2018140512, WO2018143315, WO2018206539, WO2018217651, WO2018218070, WO2019051291, WO2019099524, WO2019110751, WO2019137985, WO2019141250, CN111377918CN112159405, CN112574199, WO2021155716, WO2021197499, WO2021249563 WO2022028346, WO2022037560, WO2022068921, WO2022111521, and WO2022135591.

In some embodiments, the KRAS^{G12D} inhibitor is selected from the group consisting of MRTX1133, JAB-22000, RMC-9805 (RM-036) and pharmaceutically acceptable salts thereof.

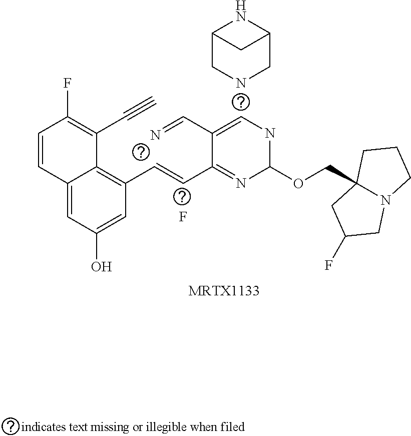

In some embodiments, the KRAS^{G13C} inhibitor is selected from the group consisting of RMC-8839 and pharmaceutically acceptable salts thereof.

In some embodiments, the KRAS^{G12V} inhibitor is selected from JAB-23000 and pharmaceutically acceptable salts thereof.

In some embodiments, the pan-KRAS inhibitor is selected from the group consisting of RMC-6236, BBP-454 and pharmaceutically acceptable salts thereof.

In some embodiments, the KRAS signaling inhibitor is a MEK inhibitor which inhibiting the mitogen-activated protein kinase 1 (MAP2KT or MEK1) and the central components of the RAS/RAF/MEK/ERK signal transduction pathway. In some embodiments, the KRAS signaling inhibitor is MEK inhibitor cobimetinib. Cobimetinib inhibits signaling downstream of all oncogenic KRAS, such as KRAS^{G12D}, KRAS^{G12V}.

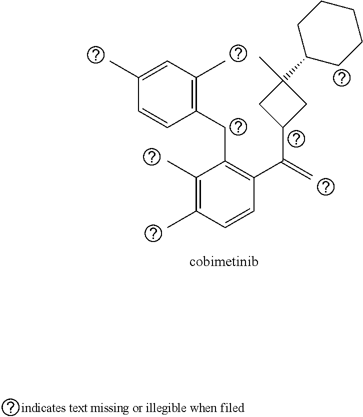

In some embodiments, the KRAS-SOS1 interaction inhibitor is selected from the group consisting of BI 1701963, BAY-293, RMC-5845, BI-3406, SDGR5 and pharmaceutically acceptable salts thereof. In some embodiments, the KRAS-SOS1 interaction inhibitors are disclosed in WO2018115380, WO2019122129, WO2018172250, WO2016077793, and WO2022017339.

The PIM kinase inhibitor and KRAS inhibitor may exist as isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses.

Exemplary isotopes that can be incorporated into compounds include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁷O, ¹⁸O, ³²P, ³³P, ³⁵S, ¹⁸F, ³⁶Cl, ¹²³I and ¹²⁵. Certain isotopically-labeled compounds (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy.

In certain embodiments, the PIM kinase inhibitor is selected from GDC-0570, GNE-1571, GNE-5775, GDC-0339 and GNE-5652 and pharmaceutically acceptable salts thereof, and the KRAS^{G12C} inhibitor is selected from sotorasib (AMG 510), ARS-853, ARS-1620, ARS-3248(JNJ-74699157), adagrasib (MRTX849), MRTX1257, LY3499446, LY3537982, BI 1823911, RG6330 (GDC-6036), RMC-6291, RMC-6236, AZD4625, D-1553, JDQ443, MK-1084, and pharmaceutically acceptable salts thereof.

### 2. Treatment of Cancer

In another aspect, the present disclosure provides a method for treating cancer to a subject in need thereof, such as a KRAS mutant cancer or KRAS inhibitor resistant cancer, comprising administering a therapeutically effective amount of a PIM kinase inhibitor and a therapeutically effective amount of a KRAS inhibitor to the subject.

In another aspect, the present disclosure provides the use of the combination of a PIM kinase inhibitor and a KRAS inhibitor in the treatment of cancer, such as KRAS mutant cancer or KRAS inhibitor resistant cancer.

In another aspect, the present disclosure provides the use of the combination of a PIM kinase inhibitor and a KRAS inhibitor in the manufacture of a medicament for use in the treatment of cancer, such as KRAS mutant cancer or KRAS inhibitor resistant cancer.

“Tumor” and “cancer” are used interchangeably herein, and refer to the physiological condition in mammals that is typically characterized by unregulated cell growth. In some embodiments, the cancer has KRAS p.G12C, G12V, G12D, G13C, G13D, Q61H, Q61L, Q61R, K117N mutation or combinations thereof. In some embodiments, the cancer has KRAS p.G12C or G12D mutation or combinations thereof.

In some embodiments, the cancer includes, but are not limited to, lung cancer, colorectal cancer, pancreatic cancer, appendix cancer, endometrial cancer, small intestine cancer, colon adenocarcinoma, rectal adenocarcinoma, small intestine adenocarcinoma, lung adenocarcinoma, non-small cell lung cancer (NSCLC), cholangiocarcinoma, plasma cell myeloma, gallbladder carcinoma, anaplastic thyroid carcinoma, ampullary cancer, cervical cancer, Gastrointestinal neuroendocrine tumor, Uterine endometrioid carcinoma, germ cell tumor, oesophagogastric cancer, bladder cancer, ovarian cancer, sex cord stromal tumor, hepatobiliary cancer, histiocytosis, anal cancer, melanoma, mature b cell neoplasms, soft-tissue sarcoma, gastrointestinal stromal tumor, head and neck cancer, glioma, prostate cancer, salivary gland cancer, breast cancer, salivary gland cancer, renal cell carcinoma, and bone cancer.

In some embodiments, the cancer is KRAS inhibitor resistant cancer. In some embodiments, the KRAS inhibitor resistant cancer includes intrinsic resistance, acquired resistance or adaptive resistance. The “KRAS resistant cancer” comprises cancers that have acquired resistance to one or more KRAS inhibitors by prior treatment with one or more KRAS inhibitors, or may comprise cancers with intrinsic resistance to one or more KRAS inhibitors.

In some embodiments, the acquired KRAS resistance is induced by KRAS inhibitor. In some embodiments, the acquired KRAS resistance is induced by sotorasib (AMG 510), ARS-853, ARS-1620, ARS-3248(JNJ-74699157), adagrasib (MRTX849), MRTX1257, LY3499446, LY3537982, BI 1823911, RG6330 (GDC-6036), RMC-6291, RMC-6236, AZD4625, D-1553, JDQ443, MK-1084, and pharmaceutically acceptable salts thereof. In some embodiments, the KRAS inhibitor resistant cancer is sotorasib (AMG 510) resistant cancer, or adagrasib (MRTX849) resistant cancer. The resistant cancer may be resistant at the beginning of treatment, or it may become resistant during treatment. In some embodiments, the sotorasib-resistance is induced by adagrasib.

In some embodiments, the KRAS inhibitor resistant cancer is selected from the group consisting of lung cancer, colorectal cancer, pancreatic cancer, appendix cancer, endometrial cancer, small intestine cancer, colon adenocarcinoma, rectal adenocarcinoma, small intestine adenocarcinoma, lung adenocarcinoma, non-small cell lung cancer (NSCLC), cholangiocarcinoma, plasma cell myeloma, gallbladder carcinoma, anaplastic thyroid carcinoma, ampullary cancer, cervical cancer, gastrointestinal neuroendocrine tumor, uterine endometrioid carcinoma, germ cell tumor, oesophagogastric cancer, bladder cancer, ovarian cancer, sex cord stromal tumor, hepatobiliary cancer, histiocytosis, anal cancer, melanoma, mature b cell neoplasms, soft-tissue sarcoma, gastrointestinal stromal tumor, head and neck cancer, glioma, prostate cancer, salivary gland cancer, breast cancer, salivary gland cancer, renal cell carcinoma, and bone cancer.

As understood herein, the PIM kinase inhibitor and the KRAS inhibitor are each administered in amounts that, in combination, are therapeutically effective. In some embodiments, the administered amounts of PIM kinase inhibitor and the KRAS inhibitor are superior effective relative to either of the monotherapy treatment. In some embodiments, the administered amounts of PIM kinase inhibitor and the KRAS inhibitor are synergistic against KRAS mutant cancer. In some embodiments, the KRAS mutant cancer has KRAS p.G12C, G12V, G12D, G13C, G13D, Q61H, Q61L, Q61R, K117N mutation or combinations thereof. In some embodiments, the cancer has KRAS p.G12C or G12D mutation or combinations thereof.

In some embodiments, the weight ratio of the PIM kinase inhibitor and the KRAS inhibitor is about 1:0.001 to 0.001:1, about 1:0.01 to about 1:100, about 1:0.1 to about 1:10, about 5:1 to about 1:5, or about 1:1 to about 1:5. In some embodiments, the weight ratio of the PIM kinase inhibitor and the KRAS inhibitor is about 1:0.01, about 10:1, about 5:1, about 3:1, about 2:1, about 1:1, about 1:0.1, about 1:0.5, about 1:1, about 1:1.5, about 1:2, about 1:3, about 1:5, about 1:10, about 1:100. In some embodiments, the weight ratio of the PIM kinase inhibitor and the KRAS inhibitor is about 500:1, about 250:1, about 200:1, about 150:1, about 120:1, about 100:1, about 30:1, about 15:1, about 14:1, about 13:1, about 12:1, about 11:1, about 10:1, about 5:1, about 3:1, about 2:1, about 1:1, about 1:0.1, about 1:0.5, about 1:1, about 1:1.5, about 1:2, about 1:3, about 1:5, about 1:10, about 1:30, about 1:100. Preferably, the KRAS inhibitor is selected from the group consisting of KRAS^{G12C} inhibitor, KRAS^{G12V} inhibitor, KRAS^{G12D} inhibitor, KRAS^{G13C} inhibitor, KRAS^{G13D} inhibitor, KRAS^{Q61H} inhibitor, KRAS^{Q61L} inhibitor, KRAS^{Q61R} inhibitor, KRAS^{K117N} inhibitor, pan-KRAS inhibitor, KRAS-SOS1 interaction inhibitor or KRAS signaling inhibitor.

In some embodiments, the weight ratio of GDC-0570 and KRAS^{G12C} inhibitor is about 1:0.001 to 0.001:1, about 1:0.01 to about 1:100, about 1:0.1 to about 1:10, about 5:1 to about 1:5, or about 1:1 to about 1:5. In some embodiments, the weight ratio of the PIM kinase inhibitor and the KRAS^{G12C} inhibitor is about 1:0.01, about 10:1, about 5:1, about 3:1, about 2:1, about 1.5:1, about 1:1, about 1:0.1, about 1:0.5, about 1:1, about 1:1.5, about 1:2, about 1:3, about 1:5, about 1:10, about 1:100. In some embodiments, the KRAS^{G12C} inhibitor is sotorasib or RG6330 (GDC-6036).

In some embodiments, the weight ratio of GDC-0570 and KRAS^{G12D} inhibitor is about 1:0.001 to 0.001:1, about 1:0.01 to about 1:100, about 1:0.1 to about 1:10, or about 1:1 to about 1:5. In some embodiments, the weight ratio of GDC-0570 and the KRAS^{G12D} inhibitor is about 500:1, about 250:1, about 200:1, about 150:1, about 120:1, about 1:0.01, about 30:1, about 15:1, about 14:1, about 13:1, about 12:1, about 11:1, about 10:1, about 5:1, about 3:1, about 2:1, about 1:1, about 1:0.1, about 1:0.5, about 1:1, about 1:1.5, about 1:2, about 1:3, about 1:5, about 1:10, about 1:30, about 1:100. In some embodiments, the KRAS^{G12D} inhibitor is MRTX1133, or KRAS^{G12D} signaling inhibitor cobimetinib. Cobimetinib is a MEK inhibitor and thereby inhibits KRAS^{G12D} signaling.

In some embodiments, the weight ratio of GDC-0570 and KRAS^{G12V} inhibitor is about 1:0.001 to 0.001:1, about 1:0.01 to about 1:100, about 1:0.1 to about 1:10, or about 1:1 to about 1:5. In some embodiments, the weight ratio of GDC-0570 and the KRAS^{G12V} inhibitor is about 500:1, about 250:1, about 200:1, about 150:1, about 120:1, about 1:0.01, about 30:1, about 15:1, about 14:1, about 13:1, about 12:1, about 11:1, about 10:1, about 5:1, about 3:1, about 2:1, about 1:1, about 1:0.1, about 1:0.5, about 1:1, about 1:1.5, about 1:2, about 1:3, about 1:5, about 1:10, about 1:100. In some embodiments, the KRAS^{G12V} inhibitor is KRAS^{G12V} signaling inhibitor cobimetinib. Cobimetinib is a MEK inhibitor and thereby inhibits KRAS^{G12V} signaling.

In some embodiments, the weight ratio of GDC-0570 and KRAS signaling inhibitor is about 1:0.001 to 0.001:1, about 1:0.01 to about 1:100, about 1:0.1 to about 1:10, or about 1:1 to about 1:5. In some embodiments, the weight ratio of GDC-0570 and the KRAS signaling inhibitor is about 500:1, about 250:1, about 200:1, about 150:1, about 120:1, about 1:0.01, about 30:1, about 15:1, about 14:1, about 13:1, about 12:1, about 11:1, about 10:1, about 5:1, about 3:1, about 2:1, about 1:1, about 1:0.1, about 1:0.5, about 1:1, about 1:1.5, about 1:2, about 1:3, about 1:5, about 1:10, about 1:100. In some embodiments, the KRAS signaling inhibitor is cobimetinib. Cobimetinib is a MEK inhibitor and inhibits signaling downstream of all oncogenic KRAS, such as KRAS^{G12D}, KRAS^{G12V}.

In some embodiments, the molar ratio of the PIM kinase inhibitor and the KRAS inhibitor is about 1:1000, about 1:100, about 1:10, or about 1:5. In some embodiments, the molar ratio of the PIM kinase inhibitor and the KRAS inhibitor is about 1:0.001 to about 1:1000, about 1:0.01 to about 1:100, about 1:0.1 to about 1:10, or about 1:1 to about 1:5.

In some embodiments, the PIM kinase inhibitor and the KRAS inhibitor are administered simultaneously. In some embodiments, the PIM kinase inhibitor and the KRAS inhibitor are administered sequentially. When administered sequentially, the combination may be administered in two or more administrations. The combined administration includes co-administration, using separate formulation, and consecutive administration in either order, wherein preferably there is a time period while both (or all) active agents simultaneously exert their biological activities.

Suitable dosages for any of the above co-administered agents are those presently used and may be lowered due to the combined action (synergy) of the newly identified agent and other chemotherapeutic agents or treatments, such as to increase the therapeutic index or mitigate toxicity or other side-effects or consequences.

In a further embodiment, the method may further comprise surgical therapy and/or radiotherapy. The amounts of the PIM kinase inhibitor, the KRAS inhibitor and the other pharmaceutically active chemotherapeutic agent(s) and the relative timings of administration will be selected in order to achieve the desired combined therapeutic effect.

In some embodiments, the present disclosure provides a method for treating cancer with KRAS mutation in a human subject in need thereof, comprising administering therapeutically effective amounts of N-(5-((2S,5R,6S)-5-amino-6-fluorooxepan-2-yl)-1-methyl-1H-pyrazol-4-yl)-2-(2,6-difluorophenyl)thiazole-4-carboxamide (GDC-0570) or a pharmaceutically acceptable salt thereof. In some embodiments, the present disclosure provides GDC-0570 or a pharmaceutically acceptable salt thereof for use as a medicament for treating cancer with KRAS mutation. In some embodiments, the present disclosure provides a use GDC-0570 or a pharmaceutically acceptable salt thereof in the manufacture of a medicament for treating cancer with KRAS mutation. In some embodiments, the GDC-0570 could be used as a single active agent in various KRAS mutant cancers. In some embodiments, the KRAS mutation is selected from G12C, G12V, G12D, G13C, G13D, Q61H, Q61L, Q61R, K117N mutation. In some embodiments, the cancer is KRAS inhibitor resistant, the KRAS inhibitor resistant comprising intrinsic resistance, acquired resistance or adaptive resistance. In some embodiments, the cancer is selected from the group consisting of lung cancer, colorectal cancer, pancreatic cancer, appendix cancer, endometrial cancer, small intestine cancer, colon adenocarcinoma, rectal adenocarcinoma, small intestine adenocarcinoma, lung adenocarcinoma, non-small cell lung cancer (NSCLC), cholangiocarcinoma, plasma cell myeloma, gallbladder carcinoma, anaplastic thyroid carcinoma, ampullary cancer, cervical cancer, gastrointestinal neuroendocrine tumor, uterine endometrioid carcinoma, germ cell tumor, oesophagogastric cancer, bladder cancer, ovarian cancer, sex cord stromal tumor, hepatobiliary cancer, histiocytosis, anal cancer, melanoma, mature b cell neoplasms, soft-tissue sarcoma, gastrointestinal stromal tumor, head and neck cancer, glioma, prostate cancer, salivary gland cancer, breast cancer, salivary gland cancer, renal cell carcinoma, and bone cancer. In some embodiments, the KRAS inhibitor resistant cancer is selected from the group consisting of lung cancer, colorectal cancer, pancreatic cancer, appendix cancer, endometrial cancer, small intestine cancer, colon adenocarcinoma, rectal adenocarcinoma, small intestine adenocarcinoma, lung adenocarcinoma, non-small cell lung cancer (NSCLC), cholangiocarcinoma, plasma cell myeloma, gallbladder carcinoma, anaplastic thyroid carcinoma, ampullary cancer, cervical cancer, gastrointestinal neuroendocrine tumor, uterine endometrioid carcinoma, germ cell tumor, oesophagogastric cancer, bladder cancer, ovarian cancer, sex cord stromal tumor, hepatobiliary cancer, histiocytosis, anal cancer, melanoma, mature b cell neoplasms, soft-tissue sarcoma, gastrointestinal stromal tumor, head and neck cancer, glioma, prostate cancer, salivary gland cancer, breast cancer, salivary gland cancer, renal cell carcinoma, and bone cancer.

### 3. Combinations, Pharmaceutical Compositions and Kit

In another aspect, the present disclosure provides a combination comprising a PIM kinase inhibitor and a KRAS inhibitor. Preferably, the combination is for use in the treatment of cancer, such as KRAS mutant cancer or KRAS inhibitor resistant cancer.

In certain embodiments, the combination is provided in a single pharmaceutical composition along with a pharmaceutically acceptable excipient. In other embodiments, the combination is provided in two pharmaceutical compositions, one comprising a PIM kinase inhibitor and pharmaceutically acceptable excipient, and another comprising a KRAS inhibitor and a pharmaceutically acceptable excipient, administered together in combination.

As used herein, pharmaceutically acceptable excipient refers to a substance that assists in the in vivo delivery and/or manufacture of a pharmaceutical composition containing the active agent or agents as described herein. Pharmaceutically acceptable excipients are inert. Non-limiting examples of pharmaceutically acceptable excipients include pharmaceutically acceptable polymers, water, NaCl, normal saline solutions, normal sucrose, normal glucose, binders, fillers, disintegrants, lubricants, surfactants, coatings, sweeteners, flavors, salt solutions, alcohols, oils, gelatins, carbohydrates, colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like. Pharmaceutically acceptable excipients are described in the Handbook of Pharmaceutical Excipients, 8^{th} Edition, published by the Pharmaceutical Press (2017), and in the United States Food and Drug Administration Inactive Ingredient Database (July 2017), the disclosures of which are incorporated by reference herein.

The pharmaceutical composition may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical compositions may be prepared for various routes and types of administration. The pharmaceutical compositions will be dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In certain embodiments, the pharmaceutical composition is formulated for oral delivery. Formulations of the PIM kinase inhibitor and/or the KRAS inhibitor suitable for oral administration may be prepared as discrete units such as pills, hard or soft e.g., gelatin capsules, cachets, troches, lozenges, aqueous or oil suspensions, dispersible powders or granules, emulsions, syrups or elixirs each containing a predetermined amount of the PIM kinase inhibitor and/or the KRAS inhibitor. The amount of compound of the PIM kinase inhibitor and the KRAS inhibitor may be formulated in a pill, capsule, solution or suspension as a combined formulation. Alternatively, the PIM kinase inhibitor and the KRAS inhibitor may be formulated separately in a pill, capsule, solution or suspension for administration by alternation.

In embodiments, the pharmaceutical composition is a solid dosage form, such as a tablet, capsule, or pill, administered orally. In embodiments, the solid dosage form is a tablet.

A dose may be administered once a day (QD), twice per day (BID), or more frequently, depending on the pharmacokinetic (PK) and pharmacodynamic (PD) properties, including absorption, distribution, metabolism, and excretion of the particular compound. In addition, toxicity factors may influence the dosage and administration dosing regimen. When administered orally, the pill, capsule, or tablet may be ingested twice daily, daily or less frequently such as weekly or once every two or three weeks for a specified period of time. The regimen may be repeated for a number of cycles of therapy.

In another aspect, the present disclosure provides a kit comprising a PIM kinase inhibitor, a KRAS inhibitor and a pharmaceutical acceptable excipient.

The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold compounds or pharmaceutically acceptable salt thereof, or a formulation thereof which is effective for treating the condition and may have a sterile access port (for example, the container may be an intravenous solution bag or a vial having a stopper pierceable by a hypodermic injection needle. At least one active agent in the composition is a compound of the PIM kinase inhibitor and/or the PIM kinase inhibitor. The label or package insert indicates that the composition is used for treating the condition of choice. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the PIM kinase inhibitor and the PIM kinase inhibitor. For example, if the kit comprises a first composition comprising the PIM kinase inhibitor and the PIM kinase inhibitor, the kit may further comprise directions for the simultaneous, sequential or separate administration of the PIM kinase inhibitor and the KRAS inhibitor to a subject in need thereof.

In another case, the kits are suitable for the delivery of solid oral forms of the PIM kinase inhibitor and/or the PIM kinase inhibitor, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

A kit may comprise (a) a first container with a PIM kinase inhibitor contained therein; and (b) a second container with a KRAS inhibitor contained therein. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises a composition of a PIM kinase inhibitor and a PIM kinase inhibitor, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXEMPLIFICATION

The present disclosure is further illustrated and explained by referring to the following examples. It should be noted that, the following examples are merely illustrative but not aims to limit the scope of the disclosure in any manner.

The various PDX models of present disclosure were established at GenenDesignCo., Ltd (Shanghai, China). The various PDX models used in the Examples are summarized in Table 1.

TABLE 1
List of PDX Models

| Example | Model ID | Cancer Type | KRAS status |
|---|---|---|---|
| Example 1 | CRC024 | Colorectal cancer | KRAS G12C |
| Example 2 | LUN055 | NSCLC | KRAS G12C |
| Example 3 | LUN156 | NSCLC | KRAS G12C |
| Example 6 | | | |
| Example 4 | LUN2156-44 | NSCLC | KRAS G12C |
| Example 5 | CRC022 | Colorectal cancer | KRAS G12C |
| Example 7 | LUN#137 | NSCLC | KRAS G12D |
| Example 8-9 | PAN092 | Pancreatic cancer | KRAS G12D |
| Example 10 | CRC051 | Colorectal cancer | KRAS G12V |

### Example 1. GDC-0570 and Sotorasib Combination Study in Sotorasib-Sensitive KRAS G12C Mutant Colorectal Cancer Patient-Derived Xenograft (PDX) Model CRC024

This study was conducted to evaluate the in vivo combinatorial antitumor efficacy of GDC-0570 and its combination with sotorasib in KRAS G12C mutant colorectal cancer (CRC) patient-derived xenograft (PDX) model CRC024. CRC024 PDX model was a sotorasib sensitive model, which was derived from a 75-year-old female Chinese CRC cancer patient. The KRAS G12C mutation in CRC PDX model was confirmed by whole exome sequencing and PCR sequencing. Tumor-bearing mice were divided into 4 groups, including vehicle, GDC-0570 at 300 mg/kg, sotorasib at 100 mg/kg, and GDC-0570 at 300 mg/kg plus sotorasib at 100 mg/kg groups. Dosing solutions were administrated orally every day. The duration of the study was 27 days. Tumor volumes were measured twice a week. Body weights were measured each day before dosing.

GDC-0570 vehicle: 0.5% Methylcellulose (MC) and 0.2% Tween-80 (MCT), kept at 2-8° C.

GDC-0570 dosing solutions: The required amount of GDC-0570 powder was weighed and mixed with appropriate amount of 0.5% MC0.2% Tween 80 solution in a container to make GDC-0570 concentration at 60 mg/ml. It was mixed via vortex and sonication until becoming homogeneous. Dosing suspension was stored at 2-8° C. for up to a week.

Sotorasib vehicle: 50% w/w Polyethylene Glycol 400 (PEG400)+50% w/w Propylene Glycol (PG).

Sotorasib dosing solutions: The required amount of sotorasib powder was weighed into a container. Mixture of 50%/50% PEG400/PG was then added into the container to make sotorasib concentration at 20 mg/ml. Solution of 1N Hydrochloric Acid was added to the same container at a final concentration of 0.39%. Sotorasib was dispersed by vortex for 5 minutes followed by sonication for 10 minutes until it was completely dissolved. Dosing solution was stored at 2-8° C. for up to a week.

### Measurement and Calculation

Tumor sizes were measured twice a week while body weights were measured daily before dosing. Clinical sign was observed on a daily basis. All animals were euthanized on day 27 after tumor size calibration. After animal euthanization, tumor samples were collected.

$$Tumor Volume ( TV ) = ( Length \times {Width}^{2} ) / 2$$

Relative Tumor Volume (RTV)=TV_{f}/TV₀, where TV₀ and TV_{f} are the tumor volume measured on day 0 and day 27, respectively;

$$Tumor Growth Inhibition T / C Ratio ( \% ) = ( RTV of the treatment group / RTV of the vehicle control group ) \times 100 \% ;$$

$$Tumor Growth Inhibition Rate ( TGI )$$

$$TGI = [ 1 - ( {TVt}_{f} - {TVt}_{0} ) / ( {TVc}_{f} - {TVc}_{0} ) ] \times 100 \%$$

TVt_{f} was the group mean tumor volume (TV) of treatment group at final treatment day
TVt₀ was the group mean TV of treatment group at treatment day 0
TVc_{f} was the group mean TV of control group at final treatment day
TVc₀ was the group mean TV of control group at treatment day 0

$$Percent of tumor regression ( \% Regression ) = 100 \times ( {TV}_{0} - {TV}_{f} ) / {TV}_{0}$$

TV₀ was the group mean TV in the same group but measured at the treatment day 0
TV_{f} was the group mean TV in the same group but measured at the last treatment day

$$Significant tumor inhibition effect : TGI ( \% ) > 60 \% , and P < 0.05 ;$$

$$Non - significant tumor inhibition effect : TGI ( \% ) ⩽ 60 \% , or P > 0.05$$

### Data Analysis

Tumor growth curve was plotted using tumor volume as Y axis and time as X axis; Body weight change curve was plotted using animal body weight as Y axis and time as X axis. Data on tumor volume and body weight change in percentage were analyzed using the One Way Analysis of Variance (One Way-ANOVA) method, followed by a significance test using the Bartlett's test (p<0.05).

The criteria used to evaluate the tumor growth inhibition effect of test articles by Tumor Growth Inhibition TGI Rate:

$$Body Weight Change in Percentage ( \% BWC ) = ( BWc - BWi ) / BWi \times 100 \% , where “ c ” refers to current , “ i ” denotes initial , “ BW ” means body weight .$$

### Study Results

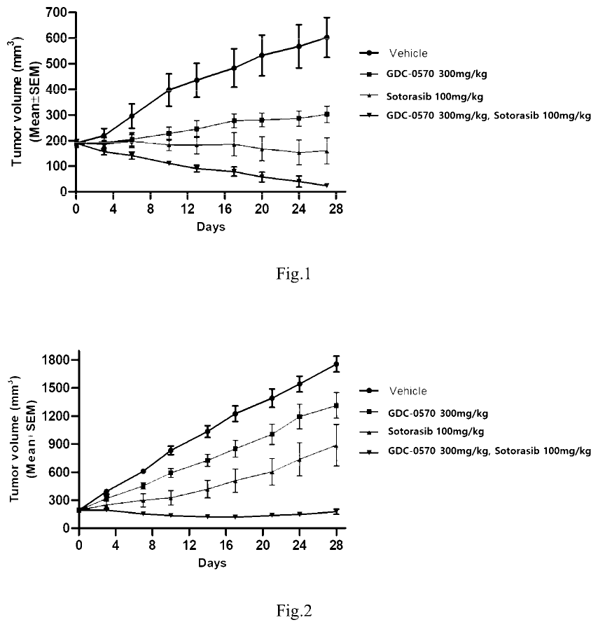

Tables 2, 3, 4 and FIG. 1 show the effect of GDC-0570 and sotorasib on tumor growth in KRAS G12C mutant CRC PDX model CRC024.

Under the current study conditions in KRAS G12C mutant CRC PDX model, GDC-0570 at 300 mg/kg, sotorasib at 100 mg/kg monotherapy treatment groups and the GDC-0570 at 300 mg/kg plus sotorasib at 100 mg/kg combination group all showed significant tumor inhibition effect. In addition, GDC-0570 and sotorasib combination group showed superior efficacy relative to either of the monotherapy treatment. In addition, GDC-0570 and sotorasib combination group showed superior efficacy relative to the monotherapy treatments.

The Mice in all dose groups did not experience severe body weight loss (defined as losing more than 20% of body weight) and also did not have other abnormalities throughout the course of study.

TABLE 2
Effect of GDC-0570 and sotorasib on tumor volume in KRAS G12C mutant CRC PDX model CRC024

| | | | | | Tumor Volume | | Mean Tumor Volume |
|---|---|---|---|---|---|---|---|
| | Test | Animal | Route of | Dose | (mm3, Mean ± SEM) | | Change (%) |
| Group | Article | Number | Administration | (mg/kg) | Day 0 | Day 27 | Day 27 vs Day 0 |
| 1 | Vehicle | 5 | p.o. | / | 190 ± 15.92 | 603 ± 78.15 | 217% |
| 2 | GDC-0570 | 5 | p.o. | 300 | 190 ± 14.81 | 303 ± 31.92 | 59% |
| 3 | sotorasib | 5 | p.o. | 100 | 190 ± 16.13 | 160 ± 50.85 | −16% |
| 4 | GDC-0570 | 5 | p.o. | 300 | 190 ± 16.17 | 23 ± 5.65 | −88% |
| | sotorasib | | p.o. | 100 | | | |

“/” denotes not applicable

TABLE 3
Summary of efficacy relative to the vehicle
in KRAS G12C mutant CRC PDX model CRC024

| | | | | | | Tumor | P Value |
|---|---|---|---|---|---|---|---|
| | Test | Animal | Dose | T/C (%) | TGI (%) | regression | (v.s. Vehicle |
| Group | Article | Number | (mg/kg) | Day 27 | Day 27 | Day 27 | Group) |
| 1 | Vehicle | 5 | / | / | / | / | / |
| 2 | GDC-0570 | 5 | 300 | 50% | 73% | no | <0.05 |
| 3 | sotorasib | 5 | 100 | 27% | >100% | 16% | <0.05 |
| 4 | GDC-0570 | | 300 | 4% | >100% | 88% | <0.05 |
| | sotorasib | 5 | 100 | | | | |

“/” denotes not applicable;
P < 0.05 means statistically significant.

TABLE 4
Summary of combination efficacy relative to monotherapies
in KRAS G12C mutant CRC PDX model CRC024

p-value

| Treatment | Dose level | vs. GDC-0570 | vs. sotorasib |
|---|---|---|---|
| group | (mg/kg) | 300 mg/kg | 100 mg/kg |
| GDC-0570 | 300 | <0.05 | <0.05 |
| sotorasib | 100 | | |

P < 0.05 means statistically significant.

### Example 2. GDC-0570 and Sotorasib Combination Study in Sotorasib-Less Sensitive KRAS G12C Mutant NSCLC Cancer Patient-Derived Xenograft (PDX) Model LUN055

This study was conducted to evaluate the in vivo combinatorial antitumor efficacy of GDC-0570 and its combination with sotorasib in KRAS G12C mutant NSCLC patient-derived xenograft (PDX) model LUN055. LUN055 PDX model was a sotorasib less-sensitive model, which was derived from a 60-year-old male Chinese NSCLC cancer patient. The KRAS^{G12C} mutation in NSCLC PDX model was confirmed by whole exome sequencing and PCR sequencing. Tumor-bearing mice were divided into 4 groups, including vehicle, GDC-0570 at 300 mg/kg, sotorasib at 100 mg/kg, and GDC-0570 at 300 mg/kg plus sotorasib at 100 mg/kg groups. Dosing solutions were administrated orally every day. The duration of the study was 28 days. Tumor volumes were measured twice a week. Body weights were measured each day before dosing.

The GDC-0570 vehicle, GDC-0570 dosing solutions, sotorasib vehicle, sotorasib dosing solutions, measurement and calculation, data analysis are the same as Example 1.

### Study Results

Tables 5-7 and FIG. 2 show the effect of GDC-0570 and sotorasib on tumor growth in KRAS G12C mutant NSCLC PDX model LUN055.

Under the current study conditions in KRAS G12C mutant NSCLC PDX model, GDC-0570 at 300 mg/kg and sotorasib at 100 mg/kg monotherapy treatment groups showed no significant tumor growth inhibition effect. In contrast combination of GDC-0570 at 300 mg/kg plus sotorasib at 100 mg/kg treatments showed significant tumor inhibition effect. In addition, GDC-0570 and sotorasib combination group showed superior efficacy relative to the monotherapy treatments.

The Mice in all dose groups did not experience severe body weight loss (defined as losing more than 20% of body weight) and also did not have other abnormalities throughout the course of study.

TABLE 5
Effect of GDC-0570 and sotorasib on tumor volume in KRAS G12C mutant NSCLC PDX model LUN055

| | | | | | Tumor Volume | | Mean Tumor Volume |
|---|---|---|---|---|---|---|---|
| | Test | Animal | Route of | Dose | (mm3, Mean ± SEM) | | Change (%) |
| Group | Article | Number | Administration | (mg/kg) | Day 0 | Day 28 | Day 28 vs Day 0 |
| 1 | Vehicle | 5 | p.o. | / | 192 ± 21.16 | 1755 ± 84.74 | 814% |
| 2 | GDC-0570 | 5 | p.o. | 300 | 192 ± 21.72 | 1313 ± 136.92 | 584% |
| 3 | sotorasib | 5 | p.o. | 100 | 192 ± 22.04 | 888 ± 222.44 | 363% |
| 4 | GDC-0570 | 5 | p.o. | 300 | 191 ± 22.04 | 171 ± 28.52 | −10% |
| | sotorasib | | p.o. | 100 | | | |

“/” denotes not applicable

TABLE 6
Summary of efficacy relative to the vehicle
in KRAS G12C mutant NSCLC PDX model LUN055

| | | | | | | Tumor | P Value |
|---|---|---|---|---|---|---|---|
| | Test | Animal | Dose | T/C (%) | TGI (%) | regression | (v.s. Vehicle |
| Group | Article | Number | (mg/kg) | Day 28 | Day 28 | Day 28 | Group) |
| 1 | Vehicle | 5 | / | / | / | / | / |
| 2 | GDC-0570 | 5 | 300 | 75% | 28% | no | >0.05 |
| 3 | sotorasib | 5 | 100 | 51% | 55% | no | <0.05 |
| 4 | GDC-0570 | 5 | 300 | 10% | >100% | 7% | <0.05 |
| | sotorasib | | 100 | | | | |

“/” denotes not applicable;
P < 0.05 means statistically significant.

TABLE 7
Summary of combination efficacy relative to monotherapies
in KRAS G12C mutant NSCLC PDX model LUN055

p-value

| Treatment | Dose level | vs. GDC-0570 | vs. sotorasib |
|---|---|---|---|
| group | (mg/kg) | 300 mg/kg | 100 mg/kg |
| GDC-0570 | 300 | <0.05 | <0.05 |
| sotorasib | 100 | | |

P < 0.05 means statistically significant.

### Example 3. GDC-0570 and Sotorasib Combination Prevented Regrowth of Tumors after Treatment Ended on Day 28 in Sotorasib-Sensitive KRAS G12C Mutant NSCLC Cancer Patient-Derived Xenograft (PDX) Model LUN156

This study was conducted to evaluate the in vivo antitumor efficacy of GDC-0570 and sotorasib in the NSCLC PDX model LUN156. LUN156 PDX model was a sotorasib sensitive model, which was derived from a 73-year-old male Chinese NSCLC cancer patient. The KRAS G12C mutation in LUN156 was confirmed by whole exome sequencing and PCR sequencing.

Tumor-bearing mice were divided into 4 groups, including vehicle, GDC-0570 at 300 mg/kg, sotorasib at 100 mg/kg, GDC-0570 at 300 mg/kg plus sotorasib at 100 mg/kg. Dosing solutions were administrated orally every day until day 28. Dosing was stopped after day 28 and tumor growth was observed continuously until day 45. Tumor volumes were measured twice a week. Bodyweights were measured each day before dosing.

The GDC-0570 vehicle, GDC-0570 dosing solutions, sotorasib vehicle, sotorasib dosing solutions, measurement and calculation, data analysis were similar to Example 1.

### Study Results

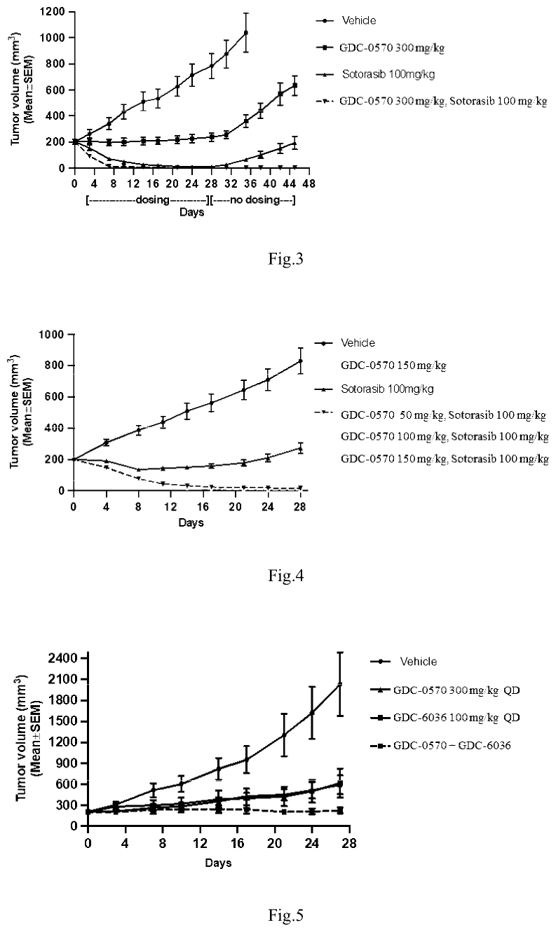

Tables 8-11 and FIG. 3 show the effect of GDC-0570 and sotorasib on tumor growth in the sotorasib-sensitive KRAS G12C mutant NSCLC PDX model.

Compared to the vehicle group, GDC-0570 at 300 mg/kg, sotorasib at 100 mg/kg monotherapy treatment groups and their combination treatment all showed significant tumor growth inhibition effect (Tables 8-11). GDC-0570 and sotorasib combination group showed superior efficacy relative to GDC-0570 monotherapy treatment that was statistically significant (Table 10). Although the difference in TGI effect between the combination group and the sotorasib monotherapy group was not statistically significant based on the p value during the 28-day dosing period (Table 10), only 1 tumor showed complete regression in the sotorasib monotherapy group, while all (5/5) tumors completely regressed by day 28 in the combination group. Moreover, after dosing was stopped on day 28, all (5/5) tumors in both GDC-0570 and the sotorasib monotherapy groups experienced re-growth, while only 1 tiny tumor reappeared in the combination group, which stayed at a minimum volume until day 45. Therefore, the combination of GDC-0570 and sotorasib prevented re-growth of tumors that occurred with sotorasib monotherapy after dosing ended. In conclusion, GDC-0570 and sotorasib combination group showed superior efficacy relative to the monotherapy treatments.

The Mice in all dose groups did not experience severe body weight loss (defined as losing more than 20% of body weight) and also did not have other abnormalities throughout the course of study.

TABLE 8
Effect of GDC-0570 and sotorasib on tumor volume in KRAS G12C mutant NSCLC PDX model LUN156

| | | | | | | | | Mean Tumor Volume | |
|---|---|---|---|---|---|---|---|---|---|
| | | | | | | | | Change (%) | |
| | Test | Animal | Route of | Dose | Tumor Volume (mm3, Mean ± SEM) | | | Day 28 vs | Day 45 vs |
| Group | Article | Number | dosing | (mg/kg) | Day 0 | Day 28 | Day 45 | Day 0 | Day 0 |
| 1 | Vehicle | 5 | p.o. | / | 202 = 20.26 | 783 = 95.56 | 1495 = 250.42 | 288% | 640% |
| 2 | GDC-0570 | 5 | p.o. | 300 | 202 = 17.47 | 237 = 33.76 | 634 = 75.40 | 17% | 214% |
| 3 | Sotorasib | 5 | p.o. | 100 | 202 = 18.18 | 11 = 2.70 | 194 = 50.75 | −95% | −4% |
| 4 | GDC-0570 | 5 | p.o. | 300 | 202 = 18.56 | 0 = 0 | 3 = 2.70 | −100% | −99% |
| | Sotorasib | | p.o. | 100 | | | | | |

“/” denotes not applicable

TABLE 9
Summary of efficacy relative to the vehicle in KRAS G12C mutant NSCLC PDX model LUN156

| | | | | | | | | | | P Value |
|---|---|---|---|---|---|---|---|---|---|---|
| | Test | Animal | Dose | T/C (%) | | TGI (%) | | Tumor regression | | (v.s. Vehicle |
| Group | Article | Number | (mg/kg) | Day 28 | Day 45 | Day 28 | Day 45 | Day 28 | Day 45 | Group) |
| 1 | Vehicle | 5 | / | | | | | | | / |
| 2 | GDC-0570 | 5 | 300 | 30% | 42% | 94% | 67% | no | no | <0.05 |
| 3 | Sotorasib | 5 | 100 | 1% | 13% | >100% | >100% | 95% | 4% | <0.05 |
| 4 | GDC-0570 | 5 | 300 | 0% | 0% | >100% | >100% | 100% | 99% | <0.05 |
| | Sotorasib | | 100 | | | | | | | |

“/” denotes not applicable;
P < 0.05 means statistically significant.

TABLE 10
Summary of combination efficacy relative to monotherapies
in KRAS G12C mutant NSCLC PDX model LUN156

p-value (at day 28)

| Treatment | Dose level | vs. GDC-0570 | vs. sotorasib |
|---|---|---|---|
| group | (mg/kg) | 300 mg/kg | 100 mg/kg |
| GDC-0570 | 300 | <0.05 | >0.05 |
| sotorasib | 100 | | |

P < 0.05 means statistically significant.

TABLE 11
Summary of tumor re-growth in the combination group relative to
the monotherapy groups in KRAS G12C mutant NSCLC PDX model LUN156

| | Test | Animal | Dose | Number CR | Number CR | Number Regrowth |
|---|---|---|---|---|---|---|
| Group | Article | Number | (mg/kg) | Day 28 | Day 45 | Day 45 |
| 1 | Vehicle | 5 | / | 0 | 0 | 5 |
| 2 | GDC-0570 | 5 | 300 | 0 | 0 | 5 |
| 3 | sotorasib | 5 | 100 | 1 | 0 | 5 |
| 4 | GDC-0570 | 5 | 300 | 5 | 4 | 1 |
| | sotorasib | | 100 | | | |

“CR” denotes complete regression

### Example 4. GDC-0570 and Sotorasib Combination Study in KRASG12C NSCLC Models with Acquired Resistance to Sotorasib LUN2156-44

This study was conducted to evaluate the in vivo combinatorial antitumor efficacy of GDC-0570 and its combination with sotorasib in KRAS G12C mutant NSCLC patient-derived xenograft (PDX) model LUN2156-44. The LUN2156-44 PDX model was a sotorasib resistant model derived from the LUN156 PDX model. The KRAS G12C mutation in LUN2156-44 was confirmed by whole exome sequencing and PCR sequencing. Tumor-bearing mice were divided into 6 groups, including vehicle, GDC-0570 at 150 mg/kg, sotorasib at 100 mg/kg, GDC-0570 at 50 mg/kg plus sotorasib at 100 mg/kg, GDC-0570 at 100 mg/kg plus sotorasib at 100 mg/kg, and GDC-0570 at 150 mg/kg plus sotorasib at 100 mg/kg groups. Dosing solutions were administrated orally every day. The duration of the study was 28 days. Tumor volumes were measured twice a week. Bodyweights were measured each day before dosing.

The GDC-0570 vehicle, GDC-0570 dosing solutions, sotorasib vehicle, sotorasib dosing solutions, measurement and calculation, data analysis are similar to Example 1.

### Study Results

Tables 12-14 and FIG. 4 show the effect of GDC-0570 and sotorasib on tumor growth in the KRAS G12C mutant NSCLC PDX model LUN2156-44.

Compared to sotorasib-sensitive PDX model LUN156 at day 28, the sotorasib-resistant PDX model LUN2156-44 reduced sotorasib single agent activity from complete regression to 88% TGI, and reduced GDC-0570 single agent activity from stasis to <50% TGI.

The measurement results of tumor volume at day 28 showed that GDC-0570 at 150 mg/kg treatment had tumor growth inhibition (TGI) at 38% and T/C ratio (%) at 71%. The sotorasib at 100 mg/kg treatment had TGI at 88% and T/C ratio (%) at 33%. Combinations of GDC-0570 at 50 mg/kg with sotorasib at 100 mg/kg showed tumor regression at 92% and T/C ratio (%) at 16%. Combinations of GDC-0570 at 100 mg/kg with sotorasib at 100 mg/kg showed tumor regression at 95% and T/C ratio (%) at 1%. Combinations of GDC-0570 at 150 mg/kg with sotorasib at 100 mg/kg showed tumor regression at 98% and T/C ratio (%) at near 0%. Compared to the vehicle group, GDC-0570 at 150 mg/kg monotherapy treatment showed no significant TGI. Sotorasib at 100 mg/kg monotherapy treatment group and its combinations with 3 different doses of GDC-0570 showed significant tumor inhibition effect. In addition, combinations of sotorasib at 100 mg/kg with GDC-0570 at 50 mg/kg, 100 mg/kg or 150 mg/kg showed superior efficacy relative to the sotorasib 100 mg/kg monotherapy treatment. Complete regression was observed in all tested combination doses, even at 50 mg/kg GDC-0570. The combination reduced the dosage of GDC-0570 needed to achieve complete regression and produced a strong synergistic effect.

Mice in all dose groups did not experience severe body weight loss (defined as losing more than 20% of body weight) and did not have other abnormalities throughout the course of the study.

TABLE 12
Effect of GDC-0570 and sotorasib on tumor volume in NSCLC PDX model LUN2156-44

| | | | | | Tumor Volume | | Mean Tumor |
|---|---|---|---|---|---|---|---|
| | Test | Animal | Route of | Dose | (mm3, Mean ± SEM) | | Volume Change (%) |
| Group | Article | Number | Administration | (mg/kg) | Day 0 | Day 28 | Day 28 vs Day 0 |
| 1 | Vehicle | 10 | p.o. | / | 200 ± 11.20 | 828 ± 83.69 | 314% |
| 2 | GDC-0570 | 10 | p.o. | 150 | 200 ± 10.98 | 590 ± 82.63 | 195% |
| 3 | Sotorasib | 10 | p.o. | 100 | 199 ± 10.39 | 273 ± 33.69 | 37% |
| 4 | GDC-0570 | 10 | p.o. | 50 | 200 ± 10.49 | 16 ± 4.20 | −92% |
| | Sotorasib | | | 100 | | | |
| 5 | GDC-0570 | 10 | p.o. | 100 | 199 ± 10.50 | 10 ± 4.09 | −95% |
| | Sotorasib | | | 100 | | | |
| 6 | GDC-0570 | 10 | p.o. | 150 | 199 ± 10.51 | 4 ± 2.06 | −98% |
| | Sotorasib | | | 100 | | | |

“/” denotes not applicable

TABLE 13
Summary of efficacy relative to the vehicle in PDX model LUN2156-44

| | | | | | | Tumor | P Value |
|---|---|---|---|---|---|---|---|
| | Test | Animal | Dose | T/C (%) | TGI (%) | regression | (v.s. Vehicle |
| Group | Article | Number | (mg/kg) | Day 28 | Day 28 | Day 28 | Group) |
| 1 | Vehicle | 10 | / | / | / | / | / |
| 2 | GDC-0570 | 10 | 150 | 71% | 38% | no | [symbol] 0.05 |
| 3 | Sotorasib | 10 | 100 | 33% | 88% | no | <0.05 |
| 4 | GDC-0570 | 10 | 50 | 16% | >100% | 92% | <0.05 |
| | Sotorasib | | 100 | | | | |
| 5 | GDC-0570 | 10 | 100 | 1% | >100% | 95% | <0.05 |
| | Sotorasib | | 100 | | | | |
| 6 | GDC-0570 | 10 | 150 | 0% | [symbol] 100°% | 98% | <0.05 |
| | Sotorasib | | 100 | | | | |

“/” denotes not applicable:
P < 0.05 means statistically significant. [symbol]
indicates data missing or illegible when filed

TABLE 14
Summary of combination efficacy relative
to monotherapies in PDX model LUN2156-44

| | p-value |
|---|---|
| Treatment group | vs. Sotorasib 100 mg/kg QD |
| GDC-05 [symbol] 0 50 mg/kg | [symbol] 0.05 |
| QD [symbol] Sotorasib 100 mg/kg QD | |
| GDC-05 [symbol] 0 100 mg/kg | [symbol] 0.05 |
| QD [symbol] Sotorasib 100 mg/kg QD | |
| GDC-05 [symbol] 0 150 mg/kg | [symbol] 0.05 |
| QD [symbol] Sotorasib 100 mg/kg QD | |

P < 0.05 means statistically significant. [symbol]
indicates data missing or illegible when filed

### Example 5. GDC-0570 and GDC-6036 Combination Study in KRAS G12C Mutant Colorectal Cancer Patient-Derived Xenograft (PDX) Model CRC022

This study was conducted to evaluate the in vivo combinatorial antitumor efficacy of GDC-0570 and its combinations with GDC-6036 in KRAS G12C mutant CRC patient-derived xenograft (PDX) model CRC022. This PDX model was derived from a 49-year-old female Chinese CRC cancer patient. The KRAS G12C mutation in CRC #022 was confirmed by whole exome sequencing and PCR sequencing. Tumor-bearing mice were divided into 4 groups, including vehicle, GDC-6036 at 100 mg/kg QD, GDC-0570 at 300 mg/kg QD, and GDC-0570 at 300 mg/kg plus GDC-6036 at 100 mg/kg. The duration of the study was 27 days. Tumor volumes were measured twice a week. Body weights were measured each day before dosing.

The GDC-0570 vehicle, GDC-0570 dosing solutions, measurement and calculation, data analysis are similar to Example 1.

GDC-6036 vehicle: 0.5% Methocel

GDC-6036 dosing solution: Weighed the appropriate amount of GDC-6036 into a container. Added appropriate volume of 0.5% Methocel to the container to make GDC-6032 at 20 mg/ml. Vortexed and sonicated repeatedly (>1 hr) to achieve a homogenous solution. Dosing solution was stored at 2-8° C. for up to a week.

### Study Results

Tables 15-16 and FIG. 5 show the effect of GDC-0570 and GDC-6036 on tumor growth in KRAS G12C mutant colorectal PDX model.

The measurement results of tumor volume at day 27 showed that GDC-6036 at 100 mg/kg QD group had tumor growth inhibition (TGI) at 78% and T/C ratio (%) at 30%. GDC-0570 at 300 mg/kg QD group had tumor growth inhibition (TGI) at 79% and T/C ratio (%) at 29%. Combination of GDC-0570 at 300 mg/kg plus GDC-6036 at 100 mg/kg had tumor growth inhibition (TGI) at 99% and T/C ratio (%) at 11%.

Compared to the vehicle group, all monotherapy groups and combination groups showed significant tumor inhibition effect. GDC-0570 monotherapy groups showed Moderate to strong tumor inhibition effect (70-80% TGI). GDC-0570 and GDC-6036 combination treatment showed superior efficacy relative to either GDC-0570 or GDC-6036 monotherapy treatment.

Mice in all dose groups did not experience severe body weight loss (defined as losing more than 20% of body weight) and did not have other abnormalities throughout the course of study. The combination of GDC-0570 and GDC-6036 was well tolerated in mice (<5% weight loss).

TABLE 15
Effect of GDC-0570 combinations on tumor volume in CRC PDX model CRC022

| | | | | | Tumor Volume | | Mean Tumor |
|---|---|---|---|---|---|---|---|
| | Test | Animal | Route of | Dose | (mm3, Mean ± SEM) | | Volume Change (%) |
| Group | Article | Number | Administration | (mg/kg) | Day 0 | Day 27 | Day 27 vs Day 0 |
| 1 | Vehicle | 5 | PO | / | 205 ± 18.63 | 2033 ± 454.39 | 892% |
| 2 | GDC-6036 | 5 | PO | 100 | 205 ± 7.46 | 615 ± 207.34 | 200% |
| 3 | GDC-0570 | 5 | PO | 300 | 205 ± 7.66 | 592 ± 134.68 | 189% |
| 4 | GDC-0570 | 5 | PO | 300 | 205 ± 19.72 | 227 ± 42.85 | 11% |
| | GDC-6036 | | PO | 100 | | | |

“/” denotes not applicable

TABLE 16
Summary of efficacies relative to the vehicle in PDX model CRC022

| | | | | | | Tumor | P Value |
|---|---|---|---|---|---|---|---|
| | Test | Animal | Dose | T/C (%) | TGI (%) | regression | (v.s. Vehicle |
| Group | Article | Number | (mg/kg) | Day 27 | Day 27 | Day 27 | Group) |
| 1 | Vehicle | 5 | / | / | / | / | / |
| 2 | GDC-6036 | 5 | 100 | 30% | 78% | no | <0.05 |
| 3 | GDC-0570 | 5 | 300 | 29% | 79% | no | <0.05 |
| 4 | GDC-0570 | 5 | 300 | 11% | 99% | no | <0.05 |
| | GDC-6036 | | 100 | | | | |

“/” denotes not applicable:
P < 0.05 means statistically significant.

### Example 6. Dose-Dependent Single Agent Activity Study of GDC-0570 in KRAS G12C Mutant NSCLC PDX Models LUN156

This study was conducted to evaluate the in vivo antitumor efficacy of GDC-0570 in NSCLC patient-derived xenograft (PDX) model LUN156. Tumor-bearing mice were divided into 5 dose groups, including vehicle group, GDC-0570 at 50 mg/kg, GDC-0570 at 100 mg/kg, GDC-0570 at 200 mg/kg and GDC-0570 at 300 mg/kg groups. Dosing solutions were administrated orally every day. The duration of the study was 21 days. Tumor volumes were measured twice a week. Body weights were measured each day before dosing.

The GDC-0570 vehicle, measurement and calculation, data analysis are similar to Example 1.

GDC-0570 dosing solutions: 60 mg/ml GDC-0570 dosing solutions of Example 1 was further diluted with MCT to other concentrations (10 mg/ml, 20 mg/ml and 40 mg/ml). Dosing suspension was stored at 2-8° C. for up to a week.

### Study Results

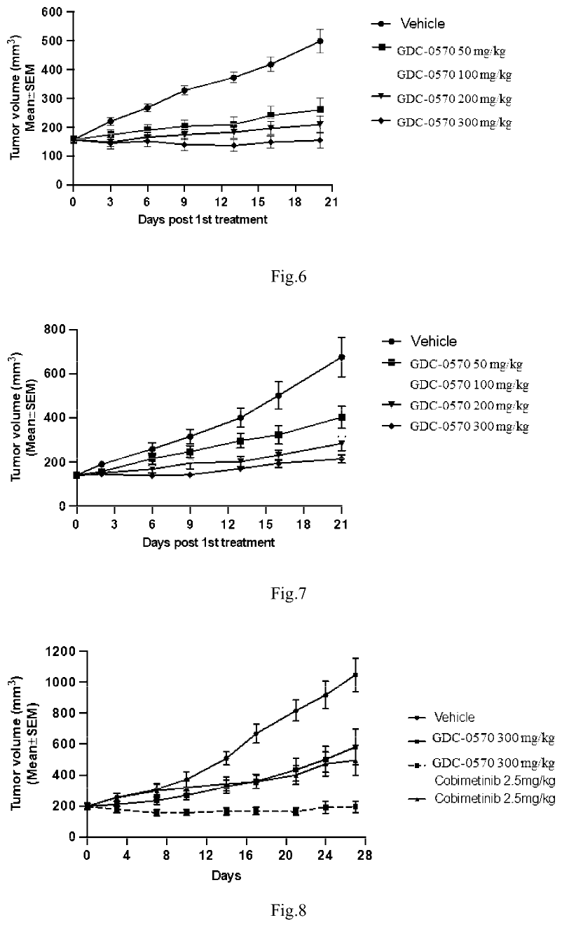

Tables 17-18 and FIG. 6 show the effect of GDC-0570 on tumor growth in KRAS G12C mutant NSCLC PDX model LUN156.

Under the current study conditions in LUN156 model, GDC-0570 at 50, 100, 200 and 300 mg/kg dosing levels all showed significantly inhibited tumor growth (TGI>60% and p-value <0.05). The mice in all dose groups did not experience severe body weight loss (defined as losing more than 20% of body weight) or other abnormalities throughout the course of study, indicating all doses of GDC-0570 were well tolerated.

TABLE 17
Effect of GDC-0570 on tumor volume in NSCLC PDX model LUN156

| | | | | Tumor Volume | | Mean Tumor |
|---|---|---|---|---|---|---|
| | Test | Animal | Dose | (mm3, Mean ± SEM) | | Volume Change (%) |
| Group | Article | Number | (mg/kg) | Day 0 | Day 22 | Day 22 vs Day 0 |
| 1 | Vehicle | 10 | / | 135 ± 9.20 | 559 ± 55.82 | 314.07% |
| 2 | GDC-0570 | 10 | 50 | 135 ± 9.09 | 338 ± 39.20 | 149.63% |
| 3 | GDC-0570 | 10 | 100 | 135 ± 9.42 | 317 ± 34.36 | 135.56% |
| 4 | GDC-0570 | 10 | 200 | 135 ± 9.65 | 314 ± 29.43 | 132.59% |
| 5 | GDC-0570 | 10 | 300 | 135 ± 9.92 | 257 ± 26.60 | 90.37% |

“/” denotes not applicable

TABLE 18
Effect of GDC-0570 on T/C Ratio
and TGI in NSCLC PDX model LUN156

| | | | | | | P Value |
|---|---|---|---|---|---|---|
| | | | | | | (v.s. |
| | Test | Animal | Dose | T/C (%) | TGI (%) | Vehicle |
| Group | Article | Number | mg/kg | Day 20 | Day 20 | Group) |
| 1 | Vehicle | 10 | / | 100% | 0% | / |
| 2 | GDC-0570 | 10 | 50 | 66.6% | 69.4% | <0.05 |
| 3 | GDC-0570 | 10 | 100 | 44.5% | 81.1% | <0.05 |
| 4 | GDC-0570 | 10 | 200 | 41.9% | 84.7% | <0.05 |
| 5 | GDC-0570 | 10 | 300 | 31.1% | 100.5% | <0.05 |

“/” denotes not applicable;
P < 0.05 means statistically significant.

### Example 7. Dose-Dependent Single Agent Activity Study of GDC-0570 in KRAS G12D Mutant NSCLC PDX Models LUN #137

This study was conducted to evaluate the in vivo antitumor efficacy of GDC-0570 in NSCLC patient-derived xenograft (PDX) model LUN #137. This PDX model was derived from a 57-year-old male Chinese NSCLC patient. The KRAS G12D mutation in LUN #137 was confirmed by whole exome sequencing and PCR sequencing.

Tumor-bearing mice were divided into 5 dose groups, including vehicle group, GDC-0570 at 50 mg/kg, GDC-0570 at 100 mg/kg, GDC-0570 at 200 mg/kg and GDC-0570 at 300 mg/kg groups. Dosing solutions were administrated orally every day. The duration of the study was 22 days. Tumor volumes were measured twice a week. Body weights were measured each day before dosing.

The GDC-0570 vehicle, measurement and calculation, data analyses are similar to Example 1. GDC-0570 dosing solutions are similar to Example 6.

### Study Results

Tables 19-20 and FIG. 7 show the effect of GDC-0570 on tumor growth in KRAS G12D mutant NSCLC PDX model LUN #137.

Under the current study conditions in LUN #137 model, GDC-0570 showed dose-dependent single agent anti-tumor activity, with significant tumor inhibitory effects observed at 200 and 300 mg/kg dose levels (TGI >60% and p-value <0.05). The mice in all dose groups did not experience severe body weight loss (defined as losing more than 20% of body weight) or other abnormalities throughout the course of study, indicating all doses of GDC-0570 were well tolerated.

TABLE 19
Effect of GDC-0570 on tumor volume in NSCLC PDX model LUN#137

| | | | Dose | Tumor Volume | | Mean Tumor Volume |
|---|---|---|---|---|---|---|
| | Test | Animal | (mg/kg) | (mm3, Mean ± SEM) | | Change (%) |
| Group | Article | Number | (mg/kg) | Day 0 | Day 21 | Day 21 vs Day 0 |
| 1 | Vehicle | 10 | / | 141 ± 10.18 | 675 ± 88.39 | 379.28% |
| 2 | GDC-0570 | 10 | 50 | 141 ± 10.67 | 403 ± 50.43 | 186.69% |
| 3 | GDC-0570 | 10 | 100 | 141 ± 11.45 | 326 ± 24.05 | 130.60% |
| 4 | GDC-0570 | 10 | 200 | 141 ± 11.46 | 285 ± 32.27 | 101.59% |
| 5 | GDC-0570 | 10 | 300 | 141 ± 11.14 | 215 ± 17.90 | 52.62% |

“/” denotes not applicable

TABLE 20
Effect of GDC-0570 on T/C Ratio and TGI in NSCLC PDX model LUN#137

| | Test | Animal | Dose | T/C (%) | TGI (%) | P Value (v.s. |
|---|---|---|---|---|---|---|
| Group | Article | Number | mg/kg | Day 21 | Day 21 | Vehicle Group) |
| 1 | Vehicle | 10 | / | 100% | 0% | / |
| 2 | GDC-0570 | 10 | 50 | 59.71% | 50.87% | >0.05 |
| 3 | GDC-0570 | 10 | 100 | 48.23% | 65.48% | >0.05 |
| 4 | GDC-0570 | 10 | 200 | 42.17% | 73.14% | <0.05 |
| 5 | GDC-0570 | 10 | 300 | 31.88% | 86.11% | <0.05 |

“/” denotes not applicable;
P < 0.05 means statistically significant.

### Example 8. GDC-0570 and Cobimetinib Combination Study in KRAS^{G12D} Pancreatic Cancer Models PAN092

This study was conducted to evaluate the in vivo combinatorial antitumor efficacy of GDC-0570 and its combinations with the MEK inhibitor cobimetinib in KRAS G12D mutant pancreatic patient-derived xenograft (PDX) model PAN092. This PDX model was derived from a 65-year-old male Chinese pancreatic cancer patient. The KRAS G12D mutation in PAN092 was confirmed by whole exome sequencing and PCR sequencing. Tumor-bearing mice were randomized into treatment groups of 5 mice each, including vehicle, cobimetinib at 2.5 mg/kg QD, GDC-0570 at 300 mg/kg QD, and GDC-0570 at 300 mg/kg plus cobimetinib at 2.5 mg/kg. The duration of the study was 27 days. Tumor volumes were measured twice a week. Body weights were measured each day before dosing.

The GDC-0570 vehicle, GDC-0570 dosing solutions, measurement and calculation, data analysis are similar to Example 1.

Cobimetinib vehicle: 0.5% methylcellulose/0.2% Tween-80 (MCT)

Cobimetinib dosing solution: Weighed the appropriate amount of cobimetinib into a container. Added appropriate volume of MCT to the container to make cobimetinib at 0.5 mg/ml. Vortexed and sonicated repeatedly to achieve a homogenous solution. Dosing solution was stored at 2-8° C. for up to a week.

### Study Results

Tables 21-23 and FIG. 8 show the effect of GDC-0570 and cobimetinib on tumor growth in KRAS G12D mutant pancreatic PDX model PAN092.

The measurement results of tumor volume at day 27 showed that cobimetinib at 2.5 mg/kg QD group had tumor growth inhibition (TGI) at 65% and T/C ratio (%) at 47%. GDC-0570 at 300 mg/kg QD group had tumor growth inhibition (TGI) at 55% and T/C ratio (%) at 56%. Combination of GDC-0570 at 300 mg/kg plus cobimetinib at 2.5 mg/kg had tumor growth inhibition (TGI) at >100% and T/C ratio (%) at 19%.

Compared to the vehicle group, all monotherapy groups and combination groups showed significant tumor inhibition effect. Cobimetinib monotherapy group showed moderate tumor inhibition effect (65% TGI). GDC-0570 monotherapy group also showed moderate tumor inhibition effect (55% TGI). GDC-0570 and cobimetinib combination treatment showed significant superior efficacy relative to either GDC-0570 or cobimetinib monotherapy treatment.

TABLE 21
Effect of GDC-0570 combinations on tumor volume in PC PDX model PAN092

| | | | | | Tumor Volume | | Mean Tumor |
|---|---|---|---|---|---|---|---|
| | Animal | Test | Route of | Dose | (mm3, Mean ± SEM) | | Volume Change (%) |
| Group | Number | Article | Administration | (mg/kg) | Day 0 | Day 27 | Day 27 vs Day 0 |
| 1 | 5 | Vehicle | p.o. | / | 199 ± 18.88 | 1046 ± 109.24 | 426% |
| 2 | 5 | GDC-0570 | p.o. | 300 | 199 ± 7.66 | 581 ± 38.59 | 192% |
| 3 | 5 | cobimetinib | p.o. | 2.5 | 198 ± 18.69 | 495 ± 96.73 | 150% |
| 4 | 5 | GDC-0570 | p.o. | 300 | 199 ± 19.17 | 195 ± 37.51 | −2% |
| | | cobimetinib | p.o. | 2.5 | | | |

“/” denotes not applicable

TABLE 22
Summary of efficacies relative to the vehicle in PDX model PAN092

| | | | | | | Tumor | P Value |
|---|---|---|---|---|---|---|---|
| | Animal | Test | Dose | T/C (%) | TGI (%) | regression | (v.s. Vehicle |
| Group | Number | Article | (mg/kg) | Day 27 | Day 27 | Day 27 | Group) |
| 1 | 5 | Vehicle | / | / | / | / | / |
| 2 | 5 | GDC-0570 | 300 | 56% | 55% | no | >0.05 |
| 3 | 5 | cobimetinib | 2.5 | 47% | 65% | no | <0.05 |
| 4 | 5 | GDC-0570 | 300 | 19% | >100% | 2% | <0.05 |
| | | cobimetinib | 2.5 | | | | |

“/” denotes not applicable; P < 0.05 means statistically significant.

TABLE 23
Summary of efficacies between treatment
groups in PDX model PAN092

| Group | Comparator Group | p value |
|---|---|---|
| GDC-0570 300 mg/kg QD | Vehicle | <0.05 |
| cobimetinib 2.5 mg/kg QD | Vehicle | <0.05 |
| GDC-0570 300 mg/kg QD + | Vehicle | <0.05 |
| cobimetinib 2.5 mg/kg QD | | |
| GDC-0570 300 mg/kg QD + | GDC-0570 300 mg/kg QD | <0.05 |
| cobimetinib 2.5 mg/kg QD | cobimetinib 2.5 mg/kg QD | <0.05 |

P < 0.05 means statistically significant.

### Example 9. GDC-0570 and MRTX1133 Combination Study in KRAS^{G12D} Pancreatic Cancer Models PAN092

This study was conducted to evaluate the in vivo combinatorial antitumor efficacy of GDC-0570 and its combinations with the KRAS G12D inhibitor MRTX1133 in KRAS G12D mutant pancreatic patient-derived xenograft (PDX) model PAN092. This PDX model was derived from a 65-year-old male Chinese pancreatic cancer patient. The KRAS G12D mutation in PAN092 was confirmed by whole exome sequencing and PCR sequencing. Tumor-bearing mice were randomized into treatment groups of 5 mice each, including vehicle, MRTX1133 at 10 mg/kg QD, GDC-0570 at 300 mg/kg QD, and GDC-0570 at 300 mg/kg plus MRTX1133 at 10 mg/kg. The duration of the study was 27 days. Tumor volumes were measured twice a week. Body weights were measured each day before dosing.

The GDC-0570 vehicle, GDC-0570 dosing solutions, measurement and calculation, data analysis are similar to Example 1.

MRTX1133 vehicle: 10% Captisol in 50 mM citrate buffer pH5.0

MRTX1133 dosing solution: Weighed the appropriate amount of MRTX1133 into a container. Added appropriate volume of vehicle to the container to make MRTX1133 formulation at 2 mg/ml. Vortexed and sonicated repeatedly (>1 hr) to achieve a homogenous solution. Dosing solution was stored at 2-8° C. for up to a week.

### Study Results

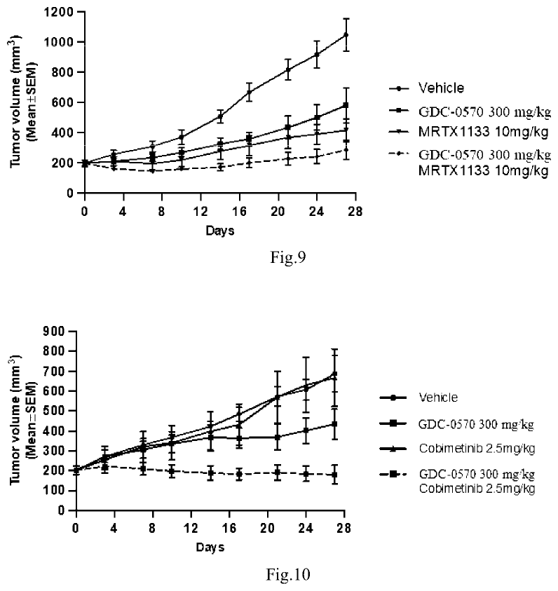

Tables 24-25 and FIG. 9 show the effect of GDC-0570 and MRTX1133 on tumor growth in KRAS G12D mutant pancreatic PDX model.

The measurement results of tumor volume at day 27 showed that MRTX1133 at 10 mg/kg QD group had tumor growth inhibition (TGI) at 74% and T/C ratio (%) at 40%. GDC-0570 at 300 mg/kg QD group had tumor growth inhibition (TGI) at 55% and T/C ratio (%) at 56%. Combination of GDC-0570 at 300 mg/kg plus MRTX1133 at 10 mg/kg had tumor growth inhibition (TGI) at 89% and T/C ratio (%) at 27%.

Compared to the vehicle group, all monotherapy groups and combination groups showed significant tumor inhibition effect. GDC-0570 and MRTX1133 monotherapy groups showed weak to moderate tumor inhibition effect (74% and 55% TGI, respectively). GDC-0570 and MRTX1133 combination treatment showed superior efficacy relative to GDC-0570 monotherapy treatment.

TABLE 24
Effect of GDC-0570 combinations on tumor volume in PC PDX model PAN092

| | | | | | Tumor Volume | | Mean Tumor |
|---|---|---|---|---|---|---|---|
| | Animal | Test | Route of | Dose | (mm3, Mean ± SEM) | | Volume Change (%) |
| Group | Number | Article | Administration | (mg/kg) | Day 0 | Day 27 | Day 27 vs Day 0 |
| 1 | 5 | Vehicle | p.o. | / | 199 ± 18.88 | 1046 ± 109.24 | 426% |
| 2 | 5 | GDC-0570 | p.o. | 300 | 199 ± 7.66 | 581 ± 38.59 | 192% |
| 3 | 5 | MRTX1133 | i.p. | 10 | 199 ± 18.80 | 415 ± 76.00 | 109% |
| 4 | 5 | GDC-0570 | p.o. | 300 | 198 ± 20.21 | 287 ± 63.37 | 45% |
| | | MRTX1133 | i.p. | 10 | | | |

“/” denotes not applicable

TABLE 25
Summary of efficacies relative to the vehicle in PDX model PAN092

| | | | | | | Tumor | P Value |
|---|---|---|---|---|---|---|---|
| | Animal | Test | Dose | T/C (%) | TGI (%) | regression | (v.s. Vehicle |
| Group | Number | Article | (mg/kg) | Day 27 | Day 27 | Day 27 | Group) |
| 1 | 5 | Vehicle | / | / | / | / | / |
| 2 | 5 | GDC-0570 | 300 | 56% | 55% | no | >0.05 |
| 3 | 5 | MRTX1133 | 10 | 40% | 74% | no | <0.05 |
| 4 | 5 | GDC-0570 | 300 | 27% | 89% | no | <0.05 |
| | | MRTX1133 | 10 | | | | |

“/” denotes not applicable; P < 0.05 means statistically significant.

### Example 10. GDC-0570 and Cobimetinib Combination Study in KRAS G12V Mutant Colorectal Cancer PDX Models CRC051

This study was conducted to evaluate the in vivo combinatorial antitumor efficacy of GDC-0570 and its combinations with cobimetinib in KRAS G12V mutant colorectal patient-derived xenograft (PDX) model CRC051. This PDX model was derived from a Chinese colorectal cancer patient. The KRAS G12V mutation in CRC051 was confirmed by whole exome sequencing and PCR sequencing. Tumor-bearing mice were divided into 4 groups, including vehicle, cobimetinib at 2.5 mg/kg QD, GDC-0570 at 300 mg/kg QD, and GDC-0570 at 300 mg/kg plus cobimetinib at 2.5 mg/kg. The duration of the study was 27 days. Tumor volumes were measured twice a week. Body weights were measured each day before dosing.

The GDC-0570 vehicle, GDC-0570 dosing solutions, measurement and calculation, data analysis are as same as Example 1.

Cobimetinib vehicle and Cobimetinib dosing solution are as same as Example 8

### Study Results

Tables 26-28 and FIG. 10 show the effect of GDC-0570 and cobimetinib on tumor growth in KRAS G12V mutant colorectal PDX model.

The measurement results of tumor volume at day 27 showed that cobimetinib at 2.5 mg/kg QD group had tumor growth inhibition (TGI) at 4% and T/C ratio (%) at 97%. GDC-0570 at 300 mg/kg QD group had tumor growth inhibition (TGI) at 52% and T/C ratio (%) at 63%. Combination of GDC-0570 at 300 mg/kg plus cobimetinib at 2.5 mg/kg had tumor growth inhibition (TGI) at >100% and T/C ratio (%) at 26%.

Compared to the vehicle group, only the combination groups showed significant tumor growth inhibition effect. Neither of the monotherapy groups show significant tumor inhibition effect.

GDC-0570 and cobimetinib combination treatment showed superior efficacy relative to either GDC-0570 or cobimetinib monotherapy treatment.

TABLE 26
Effect of GDC-0570 combinations on tumor volume in CRC PDX model CRC051

| | | | | | Tumor Volume | | Mean Tumor |
|---|---|---|---|---|---|---|---|
| | Animal | Test | Route of | Dose | (mm3, Mean ± SEM) | | Volume Change (%) |
| Group | Number | Article | Administration | (mg/kg) | Day 0 | Day 27 | Day 27 vs Day 0 |
| 1 | 5 | Vehicle | p.o. | / | 202 ± 22.28 | 688 ± 92.32 | 241% |
| 2 | 5 | GDC-0570 | p.o. | 300 | 202 ± 8.84 | 433 ± 25.16 | 114% |
| 3 | 5 | cobimetinib | p.o. | 2.5 | 202 ± 21.30 | 668 ± 145.71 | 231% |
| 4 | 5 | GDC-0570 | p.o. | 300 | 202 ± 21.45 | 180 ± 46.69 | −11% |
| | | cobimetinib | p.o. | 2.5 | | | |

“/” denotes not applicable

TABLE 27
Summary of efficacies relative to the vehicle in PDX model CRC051

| | | | | | | Tumor | P Value |
|---|---|---|---|---|---|---|---|
| | Animal | Test | Dose | T/C (%) | TGI (%) | regression | (v.s. Vehicle |
| Group | Number | Article | (mg/kg) | Day 27 | Day 27 | Day 27 | Group) |
| 1 | 5 | Vehicle | / | / | / | / | / |
| 2 | 5 | GDC-0570 | 300 | 63% | 52% | no | >0.05 |
| 3 | 5 | cobimetinib | 2.5 | 97% | 4% | no | >0.05 |
| 4 | 5 | GDC-0570 | 300 | 26% | >100% | 11% | <0.05 |
| | | cobimetinib | 2.5 | | | | |

TABLE 28
Summary of efficacies between treatment
groups in PDX model CRC051

| Group | Comparator Group | p value |
|---|---|---|
| GDC-0570 300 mg/kg QD | Vehicle | >0.05 |
| Cobimetinib 2.5 mg/kg QD | Vehicle | >0.05 |
| GDC-0570 300 mg/kg QD + | Vehicle | <0.05 |
| cobimetinib 2.5 mg/kg QD | | |
| GDC-0570 300 mg/kg QD + | GDC-0570 300 mg/kg QD | <0.05 |
| cobimetinib 2.5 mg/kg QD | cobimetinib 2.5 mg/kg QD | <0.05 |

P < 0.05 means statistically significant.

Although the foregoing has been described in some detail by way of illustration and example for purposes of clarity of understanding, the descriptions and examples should not be construed as limiting the scope of the present disclosure. All patent and scientific literature cited herein are expressly incorporated in their entirety by reference.

1. A method for treating cancer in a human subject in need thereof, comprising administering therapeutically effective amounts of a PIM kinase inhibitor and a KRAS inhibitor to the subject.

2. The method of claim 1, wherein the PIM kinase inhibitor is a PIM-1 kinase inhibitor, PIM-2 kinase inhibitor, or PIM-3 inhibitor.

3. (canceled)

4. The method of claim 2, wherein the PIM kinase inhibitor is selected from the group consisting of AZD1208, LGH447, GDC-0570, GNE-1571, GNE-5775, GDC-0339, GNE-5652 and pharmaceutically acceptable salts thereof.

5. The method of claim 3, wherein the PIM kinase inhibitor is selected from the group consisting of GDC-0570, GNE-1571, GNE-5775, GDC-0339, GNE-5652 and pharmaceutically acceptable salts thereof.

6. The method of claim 1, wherein the KRAS inhibitor is selected from the group consisting of KRAS^{G12C} inhibitor, KRAS^{G12V} inhibitor, KRAS^{G12D} inhibitor, KRAS^{G13C} inhibitor, KRAS^{G13D} inhibitor, KRAS^{Q61H} inhibitor, KRAS^{Q61L} inhibitor, KRAS^{Q61R} inhibitor, KRAS^{K117N} inhibitor, pan-KRAS inhibitor, KRAS-SOS1 interaction inhibitor, or KRAS signaling inhibitor.

7. The method of claim 6, wherein the KRAS^{G12C} inhibitor is selected from the group consisting of sotorasib (AMG 510), ARS-853, ARS-1620, ARS-3248(JNJ-74699157), adagrasib (MRTX849), MRTX1257, LY3499446, LY3537982, BI 1823911, RG6330 (GDC-6036), RMC-6291, RMC-6236, AZD4625, D-1553, JDQ443, MK-1084, and pharmaceutically acceptable salts thereof; wherein the KRAS^{G12D} inhibitor is selected from the group consisting of MRTX1133, JAB-22000, RMC-9805 (RM-036) and pharmaceutically acceptable salts thereof; wherein the KRAS^{G13C} inhibitor is selected from the group consisting of RMC-8839 and pharmaceutically acceptable salts thereof; wherein the KRAS^{G12V} inhibitor is selected from JAB-23000 and pharmaceutically acceptable salts thereof; wherein the pan-KRAS inhibitor is selected from the group consisting of and RMC-6236, BBP-454 and pharmaceutically acceptable salts thereof, wherein the KRAS-SOS1 interaction inhibitor is selected from the group consisting of BI 1701963, BAY-293, RMC-5845, BI-3406, SDGR5 and pharmaceutically acceptable salts thereof; wherein the KRAS signaling inhibitor is selected from cobimetinib and pharmaceutically acceptable salts thereof.

8. The method of claim 6, wherein the PIM kinase inhibitor is selected from the group consisting of GDC-0570, GNE-1571, GNE-5775, GDC-0339 and GNE-5652, and pharmaceutically acceptable salts thereof, and the KRAS^{G12C} inhibitor is selected from the group consisting of sotorasib (AMG 510), ARS-853, ARS-1620, ARS-3248(JNJ-74699157), adagrasib (MRTX849), MRTX1257, LY3499446, LY3537982, BI 1823911, RG6330 (GDC-6036), RMC-6291, RMC-6236, AZD4625, D-1553, JDQ443, MK-1084, and pharmaceutically acceptable salts thereof.

9. The method of claim 1, wherein the weight ratio of the PIM kinase inhibitor and the KRAS inhibitor is about 1:0.001 to 0.001:1, about 1:0.01 to about 1:100, about 1:0.1 to about 1:10, or about 1:5 to about 1:5.

10. The method of claim 1, wherein the cancer has KRAS p.G12C, G12V, G12D, G13C, G13D, Q61H, Q61L, Q61R, K117N mutation or combinations thereof.

11. The method of claim 1, wherein the cancer has KRAS p.G12C, G12D or G12V mutation or combinations thereof.

12. The method of claim 1, wherein the cancer is selected from the group consisting of lung cancer, colorectal cancer, pancreatic cancer, appendix cancer, endometrial cancer, small intestine cancer, colon adenocarcinoma, rectal adenocarcinoma, small intestine adenocarcinoma, lung adenocarcinoma, non-small cell lung cancer (NSCLC), cholangiocarcinoma, plasma cell myeloma, gallbladder carcinoma, anaplastic thyroid carcinoma, ampullary cancer, cervical cancer, gastrointestinal neuroendocrine tumor, uterine endometrioid carcinoma, germ cell tumor, oesophagogastric cancer, bladder cancer, ovarian cancer, sex cord stromal tumor, hepatobiliary cancer, histiocytosis, anal cancer, melanoma, mature b cell neoplasms, soft-tissue sarcoma, gastrointestinal stromal tumor, head and neck cancer, glioma, prostate cancer, salivary gland cancer, breast cancer, salivary gland cancer, renal cell carcinoma, and bone cancer.

13. The method of claim 1, wherein the cancer is KRAS inhibitor resistant, KRAS inhibitor sensitive or KRAS inhibitor less sensitive, the KRAS inhibitor resistant comprising intrinsic resistance, acquired resistance or adaptive resistance.

14. The method of claim 13, wherein the acquired KRAS inhibitor resistant is selected from G12C, G12V, G12D, G13C, G13D, Q61H, Q61L, Q61R, K117N inhibitor resistant.

15. The method of claim 13, wherein the acquired KRAS inhibitor resistant is selected from resistance to sotorasib (AMG 510), ARS-853, ARS-1620, ARS-3248(JNJ-74699157), adagrasib (MRTX849), MRTX1257, LY3499446, LY3537982, BI 1823911, RG6330 (GDC-6036), RMC-6291, RMC-6236, AZD4625, D-1553, JDQ443, MK-1084, MRTX1133, JAB-22000, RMC-9805 (RM-036), RMC-8839, JAB-23000, cobimetinib, RMC-6236, BBP-454, BI 1701963, BAY-293, RMC-5845, BI-3406, SDGR5 or pharmaceutically acceptable salts thereof.

16-19. (canceled)

20. A pharmaceutical composition comprising a PIM kinase inhibitor and a KRAS inhibitor and a pharmaceutical acceptable excipient, wherein the PIM kinase inhibitor is selected from the group consisting of AZD1208, LGH447, GDC-0570, GNE-1571, GNE-5775, GDC-0339, GNE-5652 and pharmaceutically acceptable salts thereof; and wherein the KRAS inhibitor is selected from the group consisting of KRAS^{G12C} inhibitor, KRAS^{G12V} inhibitor, KRAS^{G12D} inhibitor, KRAS^{G13C} inhibitor, KRAS^{G13D} inhibitor, KRAS^{Q61H} inhibitor, KRAS^{Q61L} inhibitor, KRAS^{Q61R} inhibitor, KRAS^{K117N} inhibitor, pan-KRAS inhibitor, KRAS-SOS1 interaction inhibitor, or KRAS signaling inhibitor; and
wherein the KRAS^{G12C} inhibitor is selected from the group consisting of sotorasib (AMG 510), ARS-853, ARS-1620, ARS-3248(JNJ-74699157), adagrasib (MRTX849), MRTX1257, LY3499446, LY3537982, BI 1823911, RG6330 (GDC-6036), RMC-6291, RMC-6236, AZD4625, D-1553, JDQ443, MK-1084, and pharmaceutically acceptable salts thereof; wherein the KRAS^{G12D} inhibitor is selected from the group consisting of MRTX1133, JAB-22000, RMC-9805 (RM-036) and pharmaceutically acceptable salts thereof; wherein the KRAS^{G13C} inhibitor is selected from the group consisting of RMC-8839 and pharmaceutically acceptable salts thereof; wherein the KRAS^{G12V} inhibitor is selected from JAB-23000 and pharmaceutically acceptable salts thereof; wherein the pan-KRAS inhibitor is selected from the group consisting of and RMC-6236, BBP-454 and pharmaceutically acceptable salts thereof; wherein the KRAS-SOS1 interaction inhibitor is selected from the group consisting of BI 1701963, BAY-293, RMC-5845, BI-3406, SDGR5 and pharmaceutically acceptable salts thereof; wherein the KRAS signaling inhibitor is selected from cobimetinib and pharmaceutically acceptable salts thereof.

21. (canceled)

22. The pharmaceutical composition of claim 20, wherein the pharmaceutical composition is a kit comprising the PIM kinase inhibitor, the KRAS inhibitor and a pharmaceutical acceptable excipient.

23. (canceled)

24. A method for treating cancer with KRAS mutation in a human subject in need thereof, comprising administering therapeutically effective amounts of N-(5-((2S,5R,6S)-5-amino-6-fluorooxepan-2-yl)-1-methyl-1H-pyrazol-4-yl)-2-(2,6-difluorophenyl)thiazole-4-carboxamide or a pharmaceutically acceptable salt thereof; or N-(5-((2S,5R,6S)-5-amino-6-fluorooxepan-2-yl)-1-methyl-1H-pyrazol-4-yl)-2-(2,6-difluorophenyl)thiazole-4-carboxamide or a pharmaceutically acceptable salt thereof.

25. The method of claim 24, wherein the KRAS mutation is selected from G12C, G12V, G12D, G13C, G13D, Q61H, Q61L, Q61R, and K117N mutation.

26. The method of claim 24, wherein the cancer is KRAS inhibitor resistant, and wherein the KRAS inhibitor resistant comprises intrinsic resistance, acquired resistance or adaptive resistance.

27. The method of claim 24, wherein the cancer is selected from the group consisting of lung cancer, colorectal cancer, pancreatic cancer, appendix cancer, endometrial cancer, small intestine cancer, colon adenocarcinoma, rectal adenocarcinoma, small intestine adenocarcinoma, lung adenocarcinoma, non-small cell lung cancer (NSCLC), cholangiocarcinoma, plasma cell myeloma, gallbladder carcinoma, anaplastic thyroid carcinoma, ampullary cancer, cervical cancer, gastrointestinal neuroendocrine tumor, uterine endometrioid carcinoma, germ cell tumor, oesophagogastric cancer, bladder cancer, ovarian cancer, sex cord stromal tumor, hepatobiliary cancer, histiocytosis, anal cancer, melanoma, mature b cell neoplasms, soft-tissue sarcoma, gastrointestinal stromal tumor, head and neck cancer, glioma, prostate cancer, salivary gland cancer, breast cancer, salivary gland cancer, renal cell carcinoma, and bone cancer.
